SEQUENCE LISTING (217 sequences)

SEQ ID NO: 1 - protein, 141 aa, Homo sapiens
VLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 2 - protein, 172 aa, Homo sapiens
VLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYRQAGASVAVPPARWASQRALLPSLHRPFLVFE

SEQ ID NO: 3 - protein, 172 aa, Homo sapiens
VLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYRYAGASVAVPPARWASQRALLPSLHRPFLVFE

SEQ ID NO: 4 - protein, 145 aa, Homo sapiens
VHFTAEEKAAVTSLWSKMNVEEAGGEALGRLLVVYPWTQRFFDSFGNLSSPSAILGNPKVKAHGKKVLTSFGDAIKNMDNLKPAFAKLSELHCDKLHVDENFKLLGNVMVIILATHFGKEFTPEVQAAWQKLVSAVAIALAHKYH

SEQ ID NO: 5 - protein, 146 aa, Homo sapiens
VHFTAEEKAAVTSLWSKMNVEEAGGKALGRLLVVYPWTQRFFDSFGNLSSPSAILGNPKVKAHGKKVLTSFGDAIKNMDNLKPAFAKLSELHCDKLHVDPENFKLLGNVMVIILATHFGKEFTPEVQAAWQKLVSAVAIALAHKYH

SEQ ID NO: 6 - protein, 15 aa, Homo sapiens
VHLTPEEKSAVTALW

SEQ ID NO: 7 - protein, 18 aa, Homo sapiens
TALWGKVNVDEVGGEALG

SEQ ID NO: 8 - protein, 17 aa, Homo sapiens
ALWGKVNVDEVGGEALG

SEQ ID NO: 9 - protein, 14 aa, Homo sapiens
WGKVNVDEVGGEAL

SEQ ID NO: 10 - protein, 13 aa, Homo sapiens
GKVNVDEVGGEAL

SEQ ID NO: 11 - protein, 14 aa, Homo sapiens
VDEVGGEALGRLLV

SEQ ID NO: 12 - protein, 10 aa, Homo sapiens
LVVYPWTQRF

SEQ ID NO: 13 - protein, 15 aa, Homo sapiens
RFFESFGDLSTPDAV

SEQ ID NO: 14 - protein, 14 aa, Homo sapiens
FFESFGDLSTPDAV

SEQ ID NO: 15 - protein, 18 aa, Homo sapiens
AFSDGLAHLDNLKGTFAT

SEQ ID NO: 16 - protein, 17 aa, Homo sapiens
AFSDGLAHLDNLKGTFA

SEQ ID NO: 17 - protein, 14 aa, Homo sapiens
SDGLAHLDNLKGTF

SEQ ID NO: 18 - protein, 14 aa, Homo sapiens
DGLAHLDNLKGTFA

SEQ ID NO: 19 - protein, 16 aa, Homo sapiens
DGLAHLDNLKGTFATL

SEQ ID NO: 20 - protein, 17 aa, Homo sapiens
YQKVVAGVANALAHKYH

SEQ ID NO: 21 - protein, 13 aa, Homo sapiens
VAGVANALAHKYH

SEQ ID NO: 22 - protein, 14 aa, Homo sapiens
GASVAVPPARWASQ

SEQ ID NO: 23 - protein, 13 aa, Homo sapiens
GASVAVPPARWAS

SEQ ID NO: 24 - protein, 12 aa, Homo sapiens
GASVAVPPARWA

SEQ ID NO: 25 - protein, 11 aa, Homo sapiens
GASVAVPPARW

SEQ ID NO: 26 - protein, 13 aa, Homo sapiens
ASVAVPPARWASQ

SEQ ID NO: 27 - protein, 18 aa, Homo sapiens
SVAVPPARWASQRALLPS

SEQ ID NO: 28 - protein, 12 aa, Homo sapiens
SVAVPPARWASQ

SEQ ID NO: 29 - protein, 11 aa, Homo sapiens
SVAVPPARWAS

SEQ ID NO: 30 - protein, 9 aa, Homo sapiens
SVAVPPARW

SEQ ID NO: 31 - protein, 11 aa, Homo sapiens
VAVPPARWASQ

SEQ ID NO: 32 - protein, 25 aa, Homo sapiens
AVPPARWASQRALLPSLHRPFLVFE

SEQ ID NO: 33 - protein, 17 aa, Homo sapiens
AVPPARWASQRALLPSL

SEQ ID NO: 34 - protein, 16 aa, Homo sapiens
AVPPARWASQRALLPS

SEQ ID NO: 35 - protein, 16 aa, Homo sapiens
VPPARWASQRALLPSL

SEQ ID NO: 36 - protein, 15 aa, Homo sapiens
VPPARWASQRALLPS

SEQ ID NO: 37 - protein, 10 aa, Homo sapiens
VPPARWASQR

SEQ ID NO: 38 - protein, 9 aa, Homo sapiens
VPPARWASQ

SEQ ID NO: 39 - protein, 8 aa, Homo sapiens
VPPARWAS

SEQ ID NO: 40 - protein, 18 aa, Homo sapiens
ASQRALLPSLHRPFLVFE

SEQ ID NO: 41 - protein, 15 aa, Homo sapiens
ASQRALLPSLHRPFL

SEQ ID NO: 42 - protein, 14 aa, Homo sapiens
ASQRALLPSLHRPF

SEQ ID NO: 43 - protein, 17 aa, Homo sapiens
SQRALLPSLHRPFLVFE

SEQ ID NO: 44 - protein, 14 aa, Homo sapiens
SQRALLPSLHRPFL

SEQ ID NO: 45 - protein, 13 aa, Homo sapiens
SQRALLPSLHRPF

SEQ ID NO: 46 - protein, 12 aa, Homo sapiens
SQRALLPSLHRP

SEQ ID NO: 47 - protein, 9 aa, Homo sapiens
SQRALLPSL

SEQ ID NO: 48 - protein, 16 aa, Homo sapiens
QRALLPSLHRPFLVFE

SEQ ID NO: 49 - protein, 13 aa, Homo sapiens
QRALLPSLHRPFL

SEQ ID NO: 50 - protein, 12 aa, Homo sapiens
QRALLPSLHRPF

SEQ ID NO: 51 - protein, 11 aa, Homo sapiens
QRALLPSLHRP

SEQ ID NO: 52 - protein, 15 aa, Homo sapiens
RALLPSLHRPFLVFE

SEQ ID NO: 53 - protein, 12 aa, Homo sapiens
RALLPSLHRPFL

SEQ ID NO: 54 - protein, 11 aa, Homo sapiens
RALLPSLHRPF

SEQ ID NO: 55 - protein, 10 aa, Homo sapiens
RALLPSLHRP

SEQ ID NO: 56 - protein, 14 aa, Homo sapiens
ALLPSLHRPFLVFE

SEQ ID NO: 57 - protein, 11 aa, Homo sapiens
ALLPSLHRPFL

SEQ ID NO: 58 - protein, 10 aa, Homo sapiens
ALLPSLHRPF

SEQ ID NO: 59 - protein, 9 aa, Homo sapiens
ALLPSLHRP

SEQ ID NO: 60 - protein, 13 aa, Homo sapiens
LLPSLHRPFLVFE

SEQ ID NO: 61 - protein, 12 aa, Homo sapiens
LLPSLHRPFLVF

SEQ ID NO: 62 - protein, 9 aa, Homo sapiens
LLPSLHRPF

SEQ ID NO: 63 - protein, 12 aa, Homo sapiens
LPSLHRPFLVFE

SEQ ID NO: 64 - protein, 11 aa, Homo sapiens
LPSLHRPFLVF

SEQ ID NO: 65 - protein, 10 aa, Homo sapiens
LPSLHRPFLV

SEQ ID NO: 66 - protein, 9 aa, Homo sapiens
LPSLHRPFL

SEQ ID NO: 67 - protein, 8 aa, Homo sapiens
LPSLHRPF

SEQ ID NO: 68 - protein, 7 aa, Homo sapiens
LPSLHRP

SEQ ID NO: 69 - protein, 11 aa, Homo sapiens
PSLHRPFLVFE

SEQ ID NO: 70 - protein, 7 aa, Homo sapiens
PSLHRPF

SEQ ID NO: 71 - protein, 10 aa, Homo sapiens
SLHRPFLVFE

SEQ ID NO: 72 - protein, 9 aa, Homo sapiens
LHRPFLVFE

SEQ ID NO: 73 - protein, 8 aa, Homo sapiens
HRPFLVFE

SEQ ID NO: 74 - protein, 7 aa, Homo sapiens
HRPFLVF

SEQ ID NO: 75 - protein, 7 aa, Homo sapiens
RPFLVFE

SEQ ID NO: 76 - protein, 27 aa, Homo sapiens
SVAVPPARWASQRALLPSLHRPFLVFE

SEQ ID NO: 77 - protein, 8 aa, Homo sapiens
PARWASQR

SEQ ID NO: 78 - protein, 8 aa, Homo sapiens
ALLPSLHR

SEQ ID NO: 79 - protein, 10 aa, Homo sapiens
VLSPADKTNV

SEQ ID NO: 80 - protein, 17 aa, Homo sapiens
VLSPADKTNVKAAWGKV

SEQ ID NO: 81 - protein, 19 aa, Homo sapiens
AAWGKVGAHAGEYGAEALE

SEQ ID NO: 82 - protein, 18 aa, Homo sapiens
GKVGAHAGEYGAEALERM

SEQ ID NO: 83 - protein, 12 aa, Homo sapiens
AHAGEYGAEALE

SEQ ID NO: 84 - protein, 15 aa, Homo sapiens
TYFPHFDLSHGSAQV

SEQ ID NO: 85 - protein, 14 aa, Homo sapiens
ALTNAVAHVDDMPN

SEQ ID NO: 86 - protein, 11 aa, Homo sapiens
VDDMPNALSAL

SEQ ID NO: 87 - protein, 15 aa, Homo sapiens
TLAAHLPAEFTPAVH

SEQ ID NO: 88 - protein, 14 aa, Homo sapiens
LAAHLPAEFTPAVH

SEQ ID NO: 89 - protein, 18 aa, Homo sapiens
SLDKFLASVSTVLTSKYR

SEQ ID NO: 90 - protein, 16 aa, Homo sapiens
DKFLASVSTVLTSKYR

SEQ ID NO: 91 - protein, 15 aa, Homo sapiens
KFLASVSTVLTSKYR

SEQ ID NO: 92 - protein, 12 aa, Homo sapiens
ASVSTVLTSKYR

SEQ ID NO: 93 - protein, 41 aa, Homo sapiens
VHLTPEEKSAVTALWGKVNVDEVGGKALGRLLVVYPWTQRF

SEQ ID NO: 94 - protein, 39 aa, Homo sapiens
VHLTPEEKSAVTALWGKVNVDEVGGKALGRLLVVYPWTQ

SEQ ID NO: 95 - protein, 34 aa, Homo sapiens
VHLTPEEKSAVTALWGKVNVDEVGGKALGRLLVV

SEQ ID NO: 96 - protein, 33 aa, Homo sapiens
VHLTPEEKSAVTALWGKVNVDEVGGKALGRLLV

SEQ ID NO: 97 - protein, 32 aa, Homo sapiens
VHLTPEEKSAVTALWGKVNVDEVGGKALGRLL

SEQ ID NO: 98 - protein, 31 aa, Homo sapiens
VHLTPEEKSAVTALWGKVNVDEVGGKALGRL

SEQ ID NO: 99 - protein, 29 aa, Homo sapiens
VHLTPEEKSAVTALWGKVNVDEVGGKALG

SEQ ID NO: 100 - protein, 27 aa, Homo sapiens
VHLTPEEKSAVTALWGKVNVDEVGGKA

SEQ ID NO: 101 - protein, 27 aa, Homo sapiens
LTPEEKSAVTALWGKVNVDEVGGKALG

SEQ ID NO: 102 - protein, 30 aa, Homo sapiens
TPEEKSAVTALWGKVNVDEVGGKALGRLLV

SEQ ID NO: 103 - protein, 23 aa, Homo sapiens
KSAVTALWGKVNVDEVGGKALGR

SEQ ID NO: 104 - protein, 22 aa, Homo sapiens
KSAVTALWGKVNVDEVGGKALG

SEQ ID NO: 105 - protein, 25 aa, Homo sapiens
SAVTALWGKVNVDEVGGKALGRLLV

SEQ ID NO: 106 - protein, 21 aa, Homo sapiens
SAVTALWGKVNVDEVGGKALG

SEQ ID NO: 107 - protein, 30 aa, Homo sapiens
TALWGKVNVDEVGGKALGRLLVVYPWTQRF

SEQ ID NO: 108 - protein, 28 aa, Homo sapiens
TALWGKVNVDEVGGKALGRLLVVYPWTQ

SEQ ID NO: 109 - protein, 25 aa, Homo sapiens
TALWGKVNVDEVGGKALGRLLVVYP

SEQ ID NO: 110 - protein, 23 aa, Homo sapiens
TALWGKVNVDEVGGKALGRLLVV

SEQ ID NO: 111 - protein, 22 aa, Homo sapiens
TALWGKVNVDEVGGKALGRLLV

SEQ ID NO: 112 - protein, 21 aa, Homo sapiens
TALWGKVNVDEVGGKALGRLL

SEQ ID NO: 113 - protein, 20 aa, Homo sapiens
TALWGKVNVDEVGGKALGRL

SEQ ID NO: 114 - protein, 19 aa, Homo sapiens
TALWGKVNVDEVGGKALGR

SEQ ID NO: 115 - protein, 18 aa, Homo sapiens
TALWGKVNVDEVGGKALG

SEQ ID NO: 116 - protein, 17 aa, Homo sapiens
TALWGKVNVDEVGGKAL

SEQ ID NO: 117 - protein, 16 aa, Homo sapiens
TALWGKVNVDEVGGKA

SEQ ID NO: 118 - protein, 15 aa, Homo sapiens
TALWGKVNVDEVGGK

SEQ ID NO: 119 - protein, 21 aa, Homo sapiens
ALWGKVNVDEVGGKALGRLLV

SEQ ID NO: 120 - protein, 20 aa, Homo sapiens
ALWGKVNVDEVGGKALGRLL

SEQ ID NO: 121 - protein, 18 aa, Homo sapiens
ALWGKVNVDEVGGKALGR

SEQ ID NO: 122 - protein, 17 aa, Homo sapiens
ALWGKVNVDEVGGKALG

SEQ ID NO: 123 - protein, 16 aa, Homo sapiens
ALWGKVNVDEVGGKAL

SEQ ID NO: 124 - protein, 15 aa, Homo sapiens
ALWGKVNVDEVGGKA

SEQ ID NO: 125 - protein, 14 aa, Homo sapiens
ALWGKVNVDEVGGK

SEQ ID NO: 126 - protein, 28 aa, Homo sapiens
LWGKVNVDEVGGKALGRLLVVYPWTQRF

SEQ ID NO: 127 - protein, 26 aa, Homo sapiens
LWGKVNVDEVGGKALGRLLVVYPWTQ

SEQ ID NO: 128 - protein, 23 aa, Homo sapiens
LWGKVNVDEVGGKALGRLLVVYP

SEQ ID NO: 129 - protein, 20 aa, Homo sapiens
LWGKVNVDEVGGKALGRLLV

SEQ ID NO: 130 - protein, 18 aa, Homo sapiens
LWGKVNVDEVGGKALGRL

SEQ ID NO: 131 - protein, 17 aa, Homo sapiens
LWGKVNVDEVGGKALGR

SEQ ID NO: 132 - protein, 16 aa, Homo sapiens
LWGKVNVDEVGGKALG

SEQ ID NO: 133 - protein, 15 aa, Homo sapiens
LWGKVNVDEVGGKAL

SEQ ID NO: 134 - protein, 13 aa, Homo sapiens
LWGKVNVDEVGGK

SEQ ID NO: 135 - protein, 25 aa, Homo sapiens
WGKVNVDEVGGKALGRLLVVYPWTQ

SEQ ID NO: 136 - protein, 22 aa, Homo sapiens
WGKVNVDEVGGKALGRLLVVYP

SEQ ID NO: 137 - protein, 19 aa, Homo sapiens
WGKVNVDEVGGKALGRLLV

SEQ ID NO: 138 - protein, 18 aa, Homo sapiens
WGKVNVDEVGGKALGRLL

SEQ ID NO: 139 - protein, 17 aa, Homo sapiens
WGKVNVDEVGGKALGRL

SEQ ID NO: 140 - protein, 16 aa, Homo sapiens
WGKVNVDEVGGKALGR

SEQ ID NO: 141 - protein, 15 aa, Homo sapiens
WGKVNVDEVGGKALG

SEQ ID NO: 142 - protein, 14 aa, Homo sapiens
WGKVNVDEVGGKAL

SEQ ID NO: 143 - protein, 12 aa, Homo sapiens
WGKVNVDEVGGK

SEQ ID NO: 144 - protein, 24 aa, Homo sapiens
GKVNVDEVGGKALGRLLVVYPWTQ

SEQ ID NO: 145 - protein, 21 aa, Homo sapiens
GKVNVDEVGGKALGRLLVVYP

SEQ ID NO: 146 - protein, 18 aa, Homo sapiens
GKVNVDEVGGKALGRLLV

SEQ ID NO: 147 - protein, 17 aa, Homo sapiens
GKVNVDEVGGKALGRLL

SEQ ID NO: 148 - protein, 16 aa, Homo sapiens
GKVNVDEVGGKALGRL

SEQ ID NO: 149 - protein, 15 aa, Homo sapiens
GKVNVDEVGGKALGR

SEQ ID NO: 150 - protein, 14 aa, Homo sapiens
GKVNVDEVGGKALG

SEQ ID NO: 151 - protein, 13 aa, Homo sapiens
GKVNVDEVGGKAL

SEQ ID NO: 152 - protein, 12 aa, Homo sapiens
GKVNVDEVGGKA

SEQ ID NO: 153 - protein, 11 aa, Homo sapiens
GKVNVDEVGGK

SEQ ID NO: 154 - protein, 17 aa, Homo sapiens
KVNVDEVGGKALGRLLV

SEQ ID NO: 155 - protein, 16 aa, Homo sapiens
KVNVDEVGGKALGRLL

SEQ ID NO: 156 - protein, 15 aa, Homo sapiens
KVNVDEVGGKALGRL

SEQ ID NO: 157 - protein, 14 aa, Homo sapiens
KVNVDEVGGKALGR

SEQ ID NO: 158 - protein, 13 aa, Homo sapiens
KVNVDEVGGKALG

SEQ ID NO: 159 - protein, 12 aa, Homo sapiens
KVNVDEVGGKAL

SEQ ID NO: 160 - protein, 11 aa, Homo sapiens
KVNVDEVGGKA

SEQ ID NO: 161 - protein, 10 aa, Homo sapiens
KVNVDEVGGK

SEQ ID NO: 162 - protein, 22 aa, Homo sapiens
VNVDEVGGKALGRLLVVYPWTQ

SEQ ID NO: 163 - protein, 19 aa, Homo sapiens
VNVDEVGGKALGRLLVVYP

SEQ ID NO: 164 - protein, 16 aa, Homo sapiens
VNVDEVGGKALGRLLV

SEQ ID NO: 165 - protein, 14 aa, Homo sapiens
VNVDEVGGKALGRL

SEQ ID NO: 166 - protein, 13 aa, Homo sapiens
VNVDEVGGKALGR

SEQ ID NO: 167 - protein, 12 aa, Homo sapiens
VNVDEVGGKALG

SEQ ID NO: 168 - protein, 11 aa, Homo sapiens
VNVDEVGGKAL

SEQ ID NO: 169 - protein, 10 aa, Homo sapiens
VNVDEVGGKA

SEQ ID NO: 170 - protein, 9 aa, Homo sapiens
VNVDEVGGK

SEQ ID NO: 171 - protein, 23 aa, Homo sapiens
NVDEVGGKALGRLLVVYPWTQRF

SEQ ID NO: 172 - protein, 21 aa, Homo sapiens
NVDEVGGKALGRLLVVYPWTQ

SEQ ID NO: 173 - protein, 15 aa, Homo sapiens
NVDEVGGKALGRLLV

SEQ ID NO: 174 - protein, 14 aa, Homo sapiens
NVDEVGGKALGRLL

SEQ ID NO: 175 - protein, 12 aa, Homo sapiens
NVDEVGGKALGR

SEQ ID NO: 176 - protein, 11 aa, Homo sapiens
NVDEVGGKALG

SEQ ID NO: 177 - protein, 10 aa, Homo sapiens
NVDEVGGKAL

SEQ ID NO: 178 - protein, 22 aa, Homo sapiens
VDEVGGKALGRLLVVYPWTQRF

SEQ ID NO: 179 - protein, 20 aa, Homo sapiens
VDEVGGKALGRLLVVYPWTQ

SEQ ID NO: 180 - protein, 14 aa, Homo sapiens
VDEVGGKALGRLLV

SEQ ID NO: 181 - protein, 13 aa, Homo sapiens
VDEVGGKALGRLL

SEQ ID NO: 182 - protein, 11 aa, Homo sapiens
VDEVGGKALGR

SEQ ID NO: 183 - protein, 10 aa, Homo sapiens
VDEVGGKALG

SEQ ID NO: 184 - protein, 9 aa, Homo sapiens
VDEVGGKAL

SEQ ID NO: 185 - protein, 8 aa, Homo sapiens
VDEVGGKA

SEQ ID NO: 186 - protein, 21 aa, Homo sapiens
DEVGGKALGRLLVVYPWTQRF

SEQ ID NO: 187 - protein, 19 aa, Homo sapiens
DEVGGKALGRLLVVYPWTQ

SEQ ID NO: 188 - protein, 13 aa, Homo sapiens
DEVGGKALGRLLV

SEQ ID NO: 189 - protein, 12 aa, Homo sapiens
DEVGGKALGRLL

SEQ ID NO: 190 - protein, 10 aa, Homo sapiens
DEVGGKALGR

SEQ ID NO: 191 - protein, 20 aa, Homo sapiens
EVGGKALGRLLVVYPWTQRF

SEQ ID NO: 192 - protein, 18 aa, Homo sapiens
EVGGKALGRLLVVYPWTQ

SEQ ID NO: 193 - protein, 12 aa, Homo sapiens
EVGGKALGRLLV

SEQ ID NO: 194 - protein, 11 aa, Homo sapiens
EVGGKALGRLL

SEQ ID NO: 195 - protein, 9 aa, Homo sapiens
EVGGKALGR

SEQ ID NO: 196 - protein, 7 aa, Homo sapiens
VGGKALG

SEQ ID NO: 197 - protein, 18 aa, Homo sapiens
GGKALGRLLVVYPWTQRF

SEQ ID NO: 198 - protein, 16 aa, Homo sapiens
GGKALGRLLVVYPWTQ

SEQ ID NO: 199 - protein, 12 aa, Homo sapiens
GGKALGRLLVVY

SEQ ID NO: 200 - protein, 10 aa, Homo sapiens
GGKALGRLLV

SEQ ID NO: 201 - protein, 9 aa, Homo sapiens
GGKALGRLL

SEQ ID NO: 202 - protein, 5 aa, Homo sapiens
SLHRP

SEQ ID NO: 203 - protein, 5 aa, Homo sapiens
PSLHR

SEQ ID NO: 204 - protein, 15 aa, Homo sapiens
GASVAVPPARWASQR

SEQ ID NO: 205 - protein, 10 aa, Homo sapiens
LLPSLHRPFL

SEQ ID NO: 206 - protein, 11 aa, Homo sapiens
LLPSLHRPFLV

SEQ ID NO: 207 - protein, 19 aa, Homo sapiens
VAVPPARWASQRALLPSLH

SEQ ID NO: 208 - protein, 12 aa, Homo sapiens
AVPPARWASQRA

SEQ ID NO: 209 - protein, 10 aa, Homo sapiens
AVPPARWASQ

SEQ ID NO: 210 - protein, 11 aa, Homo sapiens
AVPPARWASQR

SEQ ID NO: 211 - protein, 13 aa, Homo sapiens
SVAVPPARWASQR

SEQ ID NO: 212 - protein, 14 aa, Homo sapiens
ASVAVPPARWASQR

SEQ ID NO: 213 - protein, 16 aa, Homo sapiens
AGASVAVPPARWASQR

SEQ ID NO: 214 - protein, 16 aa, Homo sapiens
ASQRALLPSLHRPFLV

SEQ ID NO: 215 - protein, 14 aa, Homo sapiens
ALLPSLHRPFLVFE

SEQ ID NO: 216 - protein, 18 aa, Homo sapiens
AVPPARWASQRALLPSLH

SEQ ID NO: 217 - protein, 12 aa, Homo sapiens
ALLPSLHRPFLV

(19) United States
(12) Patent Application Publication
(10) Pub. No.: US 2015/0111238 A1
(43) Pub. Date: Apr. 23, 2015

(54) METHOD FOR DETECTING DISEASE BIOMARKERS

(71) Applicants: SINGAPORE HEALTH SERVICES PTE. LTD., Singapore (SG); NANYANG TECHNOLOGICAL UNIVERSITY, Singapore (SG); MAHIDOL UNIVERSITY, Singapore (SG)

(72) Inventors: Kai Tang, San Diego, CA (US); Duangmanee Sanmun, Singapore (SG); Suthat Fucharoen, Singapore (SG); Hai Yang Law, Singapore (SG); Ivy Ng, Singapore (SG)

(21) Appl. No.: 14/123,192

(22) PCT Filed: May 31, 2012
(86) PCT No.: PCT/SG2012/000195

§ 371 Date: Dec. 5, 2014

Related U.S. Application Data
(60) Provisional application No. 61/491,489, filed on May 31, 2011.

(51) Int. Cl. C12Q 1/37

(52) U.S. Cl. CPC C12Q 1/37
USPC 435/23

(57) ABSTRACT

There is presently provided a method of detecting a bio-marker for a genetic disease in an individual. The method involves comprising identifying the presence of a proteolytic peptide in a polypeptide fraction obtained from a cellular extract of cells from the individual. The cells are cells by the disease to be diagnosed, and the proteolytic peptide contains a mutation associated with the genetic disease or directly related to the disease condition.

### CROSS-REFERENCE TO RELATED APPLICATION

This application claims benefit of, and priority from, U.S. provisional application No. 61/491,489, filed on May 31, 2011, the contents of which are hereby incorporated herein by reference.

### FIELD OF THE INVENTION

The present invention relates to methods for identifying an expressed biomarker for a genetic disease.

### BACKGROUND OF THE INVENTION

Genetic testing is often used to detect genetic diseases in an individual. However, when the disease allele results from a minor mutation in the DNA, it may be more straightforward to use the expressed protein, rather than the gene, as a biomarker for disease.

With the advances of proteomic technologies, hundreds-to-thousands of candidate protein biomarkers have been identified. These markers not only provide better understanding of disease mechanisms, but also may be useful clinically as diagnostic markers.

The human plasma proteome and peptidome have been extensively exploited as providing secreted plasma proteins as protein biomarkers [1-3]. However, there are a few drawbacks to this general approach. First, not all disease-related proteins in disease cells are secreted into the bloodstream. Second, even when marker proteins or peptides are secreted into blood circulation, the existence of such secreted proteins or peptides may be difficult to confirm, given the several-order-of-magnitude higher abundance of the classical plasma proteins such as albumin and immunoglobulins. Third, many of the candidate markers discovered result from the body's response to diseases, rather than the direct cause of diseases [4], which raises the question of specificity of these candidate markers. Because of this potential lack of specificity, extensive verification processes are necessary to determine their potential clinical utility [5].

### SUMMARY OF THE INVENTION

In one aspect, the invention provides a method of detecting a biomarker for a genetic disease in an individual, the method comprising: identifying the presence of a proteolytic peptide in a polypeptide fraction, the polypeptide fraction obtained from a cellular extract of cells of the individual, which cells are affected by the disease, the proteolytic peptide containing a mutation associated with the genetic disease or directly related to the disease condition.

The mutation may comprise insertion, deletion or substitution of one or more amino acids as compared to a non-disease proteolytic peptide, or may comprise an alteration in post-translational modification as compared to a non-disease proteolytic peptide.

The method may further include one or more of the following: separating the polypeptide fraction from cellular debris in the cellular extract prior to the identifying; proteolyitcally digesting the cellular extract prior to separation of the polypeptide fraction from cellular debris or the polypeptide fraction is proteolytically digested subsequent to separation from cellular debris in the cellular extract; lysing cells from the individual that are affected by the disease to obtain the cellular extract.

In the method, identifying may comprise mass spectrometry techniques, including for example MALDI-TOF, LC/MS/MS, high resolution LC/MS, MRM or SRM techniques.

In some embodiments, the genetic disease is hemoglobinopathy or thalassernia, and the cells affected by the disease may be red blood cells. The mutation may be in an a-globin chain or in a β-globin chain. In some embodiments, the proteolytic peptide may consist of a portion of SEQ ID NO. 2 or SEQ ID NO. 3, which portion includes at least some of the sequence as defined by residues 142-172 of SEQ ID NO. 2 or SEQ ID NO. 3. In some embodiments, the proteolytic peptide consists of a sequence as set forth in any one of SEQ ID NOs. 22-78. In some embodiments, the proteolytic peptide consists of portion of SEQ ID NO. 5, which portion includes residue 26 of SEQ ID NO. 5. In some embodiments, the proteolytic peptide consists of a sequence as set forth in any one of SEQ ID NOs. 93-201. In some embodiments, the proteolytic peptide consists of a sequence as set forth in any one of SEQ ID NOs. 6-21. In some embodiments, the proteolytic peptide consists of a sequence as set forth in any one of SEQ ID NOs. 79-92.

Other aspects and features of the present invention will become apparent to those of ordinary skill in the art upon review of the following description of specific embodiments of the invention in conjunction with the accompanying figures and tables.

### BRIEF DESCRIPTION OF THE DRAWINGS

The figures and tables, which illustrate, by way of example only, embodiments of the present invention, are as follows.

FIG. 1 illustrates Homozygous CS (1A), Hb H CS (1B) and Hb H PS (1C) detected by MALDI-TOFMS. Monomers of globin chains were represented as average mass. Although the elongated a chain was very small it could be clearly detected by MALDI-TOFMS. The mass accuracy of the linear mode MALDI-TOF, at 0.1%, is enough to differentiate between α^{CS} (theoretical mass 18481.2 Da) and α^{PS} (theoretical mass 18516.2 Da).

FIG. 2 illustrates MALDI-TOF mass spectra of proteolytic peptides from different genotypes. The peptide profiles were obtained after concentrating and desalting of the hemolysates of peripheral blood samples. (A) Hb CS heterozygote (α^{CS}α/αα, β/62 ), (B) Hb CS heterozygote with α^{3.7} deletion (−^{3.7}α/α^{CS}α, β,β), (C) Hb H-CS disease (α^{CS}α/− −, β/β) (D) Hb CS homozygote (α^{CS}α/α^{CS}α, β/β) and (E) normal control.

FIG. 3 illustrates MS/MS spectra of precursor ion of 1638.76 Da and 1666.87 Da from alpha-globin CS samples. When the two peptides were subjected to collision-induced-dissociation (CID) under MALDI-TOF/TOF, peptide bonds connecting two neighboring amino acids were fragmented, generating b (N-terminal) and y (C-terminal) types of ions. Dimethylation site can be deduced from the unchanging b7 fragment (the N-terminal fragment after cleavage of the 7th peptide bond from the N-terminal, i.e. between H and R) and the mass shift of +28 Da of the b8 (the N-terminal fragment after cleavage of the 8th peptide bond from the N-terminal, i.e. between R and P), y7 (the C-terminal fragment after cleavage of the 7th peptide bond from the C-terminal, i.e. between R and H), and y8 (the C-terminal fragment after cleavage of the 8th peptide bond from the C-tenninal, i.e. between H and L) fragments.

FIG. 4 illustrates total ion chromatogram of selected Hb CS heterozygote (α^{CS}α/αα) (A), Hb CS homozygote (α^{CS}α/α^{CS}α) (B) and Hb H-CS disease (α^{CS}α/− −) (C) samples showing more proteolytic peptides were detected with increasing intensity respectively.

FIG. 5 illustrates MRM spectra obtained in the triple quadrupole mass spectrometer showing a few specific transitions selected for detection of the proteolytic fragments from the α^{CS} allele ([SEQ ID NOs. 56, 63, 71, 75]).

FIG. 6 illustrates the interface of DeNovo Explorer software, and depicts de novo sequence and MS/MS spectrum. The peak with m/z value of 2010.0940 from α^{CS 156-172}peptide was selected for CID fragmentation ([SEQ ID NOs. 57, 68, 202, 203]).

Table 1 lists proteolytic peptides detected from samples of nine genotypes, including heterozygous Hb CS, heterozygous Hb PS, homozygous Hb CS, HbH-PS disease, HbH-CS disease, compound heterozygous HbCS/alpha-thalassemia 2, triple heterozygous HbE/alpha-thalassemia 1 and HbCS, as well as normal alpha-globin type.

Table 2 lists the proteolytic peptides detected from heterozygous Hb CS, homozygous Hb CS, and Hb H-CS disease samples ([SEQ ID NOs. 26, 40-45, 48, 49, 52, 53, 57, 58, 60, 62, 69, 71, 72, 204-217]).

Table 3 lists proteolytic peptides carrying partial Hb CS sequences, detected using LC-MS from 14 different patient samples carrying the Hb CS allele. The results were filtered with the Trans Proteomic Pipeline with PeptideProphet at the 5% false discovery level ([SEQ ID NOs. 22-78]).

Table 4 lists Hb E proteolytic peptides found in several form of hemoglobinopathes including heterozygous Hb E, homozygous Hb E, and β-thalassemia/Hb E disease samples ([SEQ ID NOs. 93-201]).

### DETAILED DESCRIPTION

Disease-causing protein mutations, if known and easily detected, may serve as definitive biomarkers for a given disease due to the specificity of the mutation for the disease. However, proteins carrying such mutations are rare and often in low abundance, making them difficult to detect.

The methods as described herein relate to direct detection of disease mutations within a protein, by detecting proteolytic fragments of the mutated proteins as the proteins are degraded within the cell. The methods relate to the hypothesis that the mutant proteins involved in disease are typically unstable within the cell, and thus tend to be degraded within the cell by endogenous proteases. Proteolytic peptides carrying the disease mutation should therefore be detectable within the cell, and could serve as potential biomarkers for disease.

Previous methods have focussed on detecting protein biomarkers that have been secreted into blood plasma. However, not all disease-related proteins expressed in cells associated with disease are secreted into the bloodstream. Some of the most specific markers may still remain in the diseased cells. As well, even when biomarker proteins or peptides containing a disease mutation leak into circulation, such proteins and peptides may be difficult to isolate given the several-order-of-magnitude higher abundance of the classical plasma proteins such as albumin and immunoglobulins.

Another drawback to detecting disease protein biomarkers in blood plasma is that many of the candidate biomarkers identified in fact result from the body's response to disease, such as inflammation, angiogenesis, fibrosis, etc., and are not related to the direct cause of a specific disease [4]. Proteins involved in a response to disease may be present for more than one type of disease or disorder. This raises the question of specificity of such candidate biomarkers in the bloodstream. Thus, the use of secreted proteins as biomarkers often requires extensive verification processes to determine the potential clinical utility of the secreted biomarker [5].

The methods described herein focus on proteins and peptides contained within cells affected by the disease that is to be screened, cells that would be expressing the mutant version of the protein involved in the relevant disease for which an individual is to be screened. Accordingly, there is presently provided a method of detecting a biomarker for a genetic disease in an individual.

As used herein, disease allele, or mutant allele refer to alleles associated with or having a mutation, the mutation resulting in, causing, or being directly associated with, a disease or disorder in an individual, and which results in an expressed protein containing a mutation. Accordingly, disease protein and mutant protein refer to a protein expressed from a disease allele that contains a mutation, which mutation results in, causes or is directly associated with the disease or disorder. The mutation may be dominant or recessive, and may occur in combination with other mutations involved in disease. The mutation may be an alteration or difference in post-translational modification of a specific protein, which differently modified protein is implicated in or associated with disease.

In contrast, a non-disease allele is an allele that is not associated with disease or disorder and which results in expression of a protein that exhibits normal protein function as is found in a healthy individual not afflicted with the disease or disorder. A non-disease protein is a protein expressed from a non-disease allele. A non-disease allele or protein does not contain the mutation that results in disease associated with or directly related to a disease allele or protein, and may be referred to as a wildtype allele or protein, or healthy allele or protein.

A disease allele or protein is considered a biomarker, in that detection of a mutation associated with disease can be used to confirm that an individual carries a mutation associated with a genetic disorder, and may be at risk of developing the disease or disorder or may have the disease or disorder. A protein biomarker is a disease protein that can be used in a diagnostic method to confirm that the individual carries at least one disease allele.

The method is performed on a sample taken from an individual to be screened for the genetic disorder. The genetic disorder may be any genetic disorder that results in expression of a mutant protein, including a mutation in primary sequence or a difference in post-translational modification, as indicated above.

For example, the genetic disorder may be a disorder that arises from a point mutation, insertion mutation or deletion mutation in a gene, resulting in a defective or altered protein, leading to disease or disorder. The point mutation may result in substitution of one amino acid for another, or may result in introduction of alteration of stop codon or exon splice site and thus produce a protein that is longer or shorter, or has deleted or inserted amino acids relative to the non-disease protein. The deletion or insertion mutations in the gene may result in a frame shift partway down a coding sequence or may result in an in-frame insertion or deletion of amino acids in the coding sequence.

The genetic disorder may be a disorder that results in a change in the post-translational modification of a protein. For example, the protein may lack a modification found on a healthy or wildtype protein, may be modified at a different position relative to a healthy or wildtype protein, or may have additional modifications not found on a healthy or wildtype protein.

The genetic mutation as described above results in expression of a disease protein. The disease protein will thus have a sequence or post-translational modification that is altered from a non-disease protein, for example as expressed from an un-mutated, non-disease allele in a healthy individual. Thus, at least some proteolytic peptide fragments of the disease protein will be distinguishable from proteolytic peptide fragments generated from a non-disease protein.

A proteolytic peptide refers to a peptide fragment of a protein resulting from digestion with one or more proteases. Thus, a proteolytic peptide consists of a sequence found within the full length protein from which the proteolytic peptide is derived. Therefore, reference to a disease peptide, proteolytic disease peptide or proteolytic peptide associated with a disease or disorder is reference to a proteolytic fragment of a disease protein that contains a mutation in the amino acid sequence, namely one or more substituted, inserted or deleted amino acid, or that contain an amino acid residue that is differently post-translationally modified, compared to proteolytic peptides generated from a non-disease protein. Similarly, reference to a non-disease peptide, wildtype peptide or proteolytic peptide from a non-disease gene is reference to a proteolytic fragment of a non-disease protein, which contains a wildtype sequence, i.e. no mutation, or contains an amino acid residue that is post-translationally modified as found in the non-disease protein.

The sample obtained from an individual contains cells affected by the disease, meaning that the cells express the mutant disease protein. Thus, where the protein is expressed in a cell-specific manner, the sample will contain the cell type in which the disease protein is expressed.

The individual is any individual that is to be diagnosed for a particular genetic disease or disorder, or to be identified as a carrier of a disease allele if the disease or disorder is associated with a recessive genetic mutation. The individual may be symptomatic or asymptomatic for the disease or disorder, and may be any individual in which the disease or disorder is suspected, a predisposition to disease or disorder is suspected, or with a family history of the disease or disorder. The individual may be suspected of being homozygous or heterozygous for a disease allele.

The method is performed on peptides obtained from cells of the individual that will express the protein associated with or directly related to the disease. The peptides may include soluble peptides found in the soluble fraction of a cell lysate (also referred to as cellular extract), or may include membrane-bound peptides by first solubilising such peptides, using routine laboratory methods such as performing a detergent extraction of proteins and peptides, such that the membrane-bound proteins become soluble in the soluble fraction of the cellular extract following treatment.

Thus, in the method, cells obtained from an individual may be lysed. The cellular extract will contain proteolytic peptides, including proteolytic non-disease peptides and may also contain proteolytic disease peptides. The cellular extract may be further fractionated, as indicated below, and the fraction or portion of a cellular extract (including the whole cellular extract if not further fractionated) that contains the proteolytic peptides is considered a polypeptide fraction of the cellular extract.

Thus, if desired, cell debris may be removed in order to obtain a polypeptide cell fraction containing soluble proteins and peptides, including subsequent to a solubilization step such as detergent treatment. Alternatively, the whole cellular extract, which will contain the polypeptide fraction along with other insoluble cell debris, may be used directly as a polypeptide fraction.

Cell lysis and optional removal of debris may be achieved using standard molecular biology laboratory techniques. For example, cells may be lysed using chemical disruption, mechanical grinding, freeze/thaw, liquid homogenization, or sonication techniques. Insoluble cell debris may be separated from the soluble polypeptide fraction by centrifugation techniques. For example, cells may be lysed by suspension in a hypotonic or other lysis buffer, which may include detergent, followed by centrifugation.

If desired, protease inhibitors may be added before, during or after cell lysis to inhibit any further degradation of peptides in the cell. Protease inhibitors, including protease inhibitor cocktails are readily commercially available, and include for example general inhibitors for inhibition of serine and/or cysteine proteaSes, including inhibitors such as PMSF, Pefabloc SC, aprotinin, leupeptin, etc. For example, a complete EDTA-free protease inhibitor cocktail tablet can be purchased from Roche Applied Science.

In addition, or alternatively, particular proteases, which may be non-targets for any protease inhibitors included, may be added before, during or after cell lysis in order to target particular peptide sequences for further proteolysis. Proteolytic digestion of polypeptides in the sample may be performed subsequent to cell lysis but before any further separation of the polypeptide fraction from insoluble cell debris, or may be performed on the polypeptide fraction obtained following removal of insoluble cell debris. Specific proteases may be added in order to produce specific desired fragments of known target proteins, including specific serine or cysteine proteases.

If desired, peptides may be separated from larger soluble proteins in the soluble polypeptide fraction, for example by using size exclusion techniques. Filtration, chromatography, ultracentrifugation, precipitation, and dialysis techniques may be used to isolate smaller molecular weight peptides away from larger proteins.

Once a sample polypeptide fraction containing soluble peptides from the cells that express the biomarker protein is obtained, particular peptides within the fraction are then detected in order to confirm the presence of a proteolytic peptide fragment of a disease protein containing the mutation.

Routine laboratory methods may be used to identify proteolytic peptide fragments.

For example, techniques including gel electrophoresis, isoelectric focusing (IEF), cation exchange HPLC, or mass spectrometry may be used.

In one embodiment of the method, mass spectrometry methods are used to identify proteolytic disease peptides derived from mutant disease proteins. Such methods are efficient, accurate methods to isolate and identify biomolecules and are well suited to separation and identification of proteolytic disease peptides having a disease mutation that may differ by a single amino acid or that may differ with respect to post-translational modifications as compared to a proteolytic non-disease peptide which may be contained in the same sample of a heterozygous individual.

Thus, techniques such as MALDI-TOF mass spectrometry, LC/MS/MS (MRM) or high resolution LC/MS may be used. Preparation of the sample fraction containing the proteolytic peptides may be done in accordance with routine methods. For example, the fraction of the sample containing the proteolytic peptides may be mixed with a UV-absorbing matrix prior to laser irradiation in a mass spectrometer or injected for LC/MS.

Techniques such as tandem mass spectroscopy (MS/MS) and liquid chromatography/mass spectroscopy (LC/MS and LC/MS/MS) can be used to obtain the sequence of individual peptides in the sample. Briefly, in LC/MS, different peptides are separated by a reverse phase column according to their hydrophobicity and sprayed into a mass spectrometer for mass measurement. In LC/MS/MS, the peptide is further fragmented in the mass spectrometer to generate fragments, and one, a few, or all of the fragments can be measured in the mass spectrometer to increase specificity.

Thus, for example, the use of selected reaction monitoring (SRM) mode and multiple reaction monitoring mode (MRM) can be performed using tandem MS methods in order to identify the sequences of the particular peptides contained within the sample.

As indicated above, mass spectroscopy methods can also be used to identify differences in post-translational modification between proteolytic peptides containing a disease mutation and non-disease peptides.

If desired, the results obtained with the sample from the individual to be diagnosed may be compared with samples containing known non-disease proteolytic peptides without any mutation and/or with samples containing known disease proteolytic peptides containing known disease mutations.

An exemplified embodiment of the method is described below, although it will be appreciated that the method is not limited to the following embodiment.

In the exemplified embodiment, the disease allele is an altered hemoglobin gene. Certain mutations within the a-hemoglobin or β-hemoglobin genes result in altered proteins that give rise to hemoglobinopathy blood disorders such as α-thalassemia, β-thalassemia, sickle cell anemia or hemolyticanemia. β-hemoglobin variants include, without limitation, Hb E, Hb S, Hb C, Hb D-Punjab or Hb 0-Arab. α-hemoglobin variants include, without limitation, Hb CS, Hb PS, Hb Queens, Hb J-Buda or Hb Q-Thailand. As will be appreciated, there are many other disorders and specific mutations that have been identified for the α-hemoglobin or β-hemoglobin genes, all of which are included within the scope of the method.

Hemoglobin disorders are most prevalent in Southeast Asia. Thalassemias are characterized by a reduction or absence of haemoglobin chain (either α or β) production.

α-Thalassemia is caused by deletional or non-deletional mutations in the α-hemoglobin gene. The non-disease (i.e. wildtype) sequence for α2-hemoglobin protein is [SEQ ID NO. 1]:

VLSPADKTNV KAAWGKVGAHAGEYGAEALE RMFLSFPTTK
TYFPHFDLSH GSAQVKGHGK KVADALTNAV AHVDDMPNAL
SALSDLHAHK LRVDPVNFKL LSHCLLVTLA AHLPAEFTPA
VHASLDKFLA SVSTVLTSKYR

The Hemoglobin Constant Spring (Hb CS) genotype, the most common non-deletional α-thalassemia in Thai and Chinese, is caused by a mutation at the stop codon of the α2-hemoglobin gene, TAA→CAA, resulting in a 31-amino acid elongation of the of the α2-hemoglobin protein [6] (additional amino acids shown in boxed sequence) [SEQ ID NO. 2]:

VLSPADKTNVKAAWGKVGAHAGEYGAEALERMFLSFPTTK
TYFPHFDLSHGSAQVKGHGKKVADALTNAVAHVDDMPNAL
SALSDLHAHKLRVDPVNFKLLSHCLLVTLAAHLPAEFTPA
VHASLDKFLASVSTVLTSKYRQAGASVAVPPARWASQRALL
PSLHRPFLVFE

Another less common non-deletional type, Hemoglobin Pakse (Hb PS), also occurs from a different mutation at the same position on a2 hemoglobin allele, TAA→TAT [6, 7], again resulting in a 31-amino acid elongation of the of the α2-hemoglobin protein (additional amino acids shown in boxed sequence) [SEQ ID NO. 3]:

VLSPADKTNVKAAWGKVGAHAGEYGAEALERMFLSFPTTK
TYFPHFDLSHGSAQVKGHGKKVADALTNAVAHVDDMPNAL
SALSDLHAHKLRVDPVNFKLLSHCLLVTLAAHLPAEFTPA
VHASLDKFLASVSTVLTSKYRYAGASVAVPPARWASQRALL
PSLHRPFLVFE

In comparison with Hb H-CS disease, the clinical and hematological data in patients with Hb-PS has no significant difference [8, 9]. These α-hemoglobin elongation mutations can interact with α-thalassemia 1 causing Hb H disease, especially in Thai population [7, 8].

Molecular characterization by using mismatched polymerase chain reaction-restriction fragment length polymorphism (PCR-RFLP) has previously been applied in screening programs [9] to identify Hb CS and PS mutations. An initial diagnosis of Hb CS and PS has also been previously qualitatively performed using ion exchange high performance liquid chromatography (IE-HPLC; Bio-Rad Variant) [9, 10]. However, it is difficult to detect the peak chromatogram in its heterozygous state because of the low abnormal Hb level due to the instability of Hb CS and Hb PS-globin mRNAs and proteins. The appearance of Hb CS or Hb PS peak can lead to the diagnosis of either homozygous Hb CS or severe a-thalassemia intermediate phenotype for Hb H-CS or Hb H-PS disease [11].

Initially, reports suggested an unexpected increase of α/β ratio (>1) in Hb CS heterozygotes and homozygotes [22]. It was later demonstrated that there was a deficit of a chain production in a short period of in vitro incubation and the excess p chain was gradually lost with a longer period of 3-hour incubation, so that the α/β globin chain synthesis ratio appeared to be more than 1[23]. The erythroblastic marrow cells can degrade unstable protein quickly but the unstable globin chain that precipitate in mature erythrocytic cells will also be destroyed, leading to a greater amount of circulating polypeptides of hemoglobin [24]. Additionally, minute amounts of the α^{CS} hemoglobin may still remain in peripheral blood as it can be detected in homozygous α^{CS} by HPLC.

Therefore, the present method was performed using red blood cells from individuals, on the basis that minor globin proteins (intact form of Hb variants) could be detected directly by mass spectrometry without HPLC separation, as described in Example 1 below. Moreover, due to proteolysis of the elongated hemoglobin chain, peptide fragments may be found as well by mass spectrometric analysis.

Thus, the methods as described herein can be used to directly detect intact Hbα^{CS} and α^{PS} peptide biomarkers from peripheral blood samples, for example by using matrix assisted laser desorption/ionization time-of-flight(MALDI-TOF) mass spectrometry as described in Example 1 below. Furthermore, proteolytic peptide fragments can be found in red cellular extract and sequenced using tandem mass spectrometry. The proteolytic disease peptides have been identified to originate from the elongated C-terminal sequence, thus including peptides having any portion of [SEQ ID NO. 2] or [SEQ ID NO. 3] that encompasses at least part of the extended C-terminal region of the α2-globin gene, i.e. the part defined by amino acids 142-172 of [SEQ ID NO. 2] or [SEQ ID NO. 3].

In the β-hemoglobin gene, the non-disease sequence is as follows [SEQ ID NO. 4]:

VHFTAEEKAAVTSLWSKMNVEEAGGEALGRLLVVYPWTQRFFDSFG
NLSSPSAILGNPKVKAHGKKVLTSFGDAIKNMDNLKPAFAKLSELH
CDKLHVDENFKLLGNVMVIILATHFGKEFTPEVQAAWQKLVSAVAI
ALAHKYH

The Hb E mutation results in single amino acid change E→K at position 26 in the β-hemoglobin protein (the substituted amino acid is indicated in bold and underline) [SEQ ID NO. 5]:

VHFTAEEKAAVTSLWSKMNVEEAGGKALGRLLVVYPWTQRFFDSFG
NLSSPSAILGNPKVKAHGKKVLTSFGDAIKNMDNLKPAFAKLSELH
CDKLHVDPENFKLLGNVMVIILATHFGKEFTPEVQAAWQKLVSAVA
IALAHKYH

Heterozygous Hb E and homozygous Hb E are asymptomatic, but the compound heterozygote with Hb E and β-thalassemia usually exhibits a diseased phenotype. Therefore, prevention and control of thalassemias should be considered [37], since Hb E is an abnormal hemoglobin with reduced synthesis. Instability of Hb E was also observed in Hb E patients [38]. The dichlorophenolindophenol (DCIP) test was used to detect for unstable hemoglobin such as Hb E. The DCIP test is a rapid and sensitive screening technique. Thus, the diagnostic methods as described herein can be used as an accurate approach to screening and diagnosis for Hb E patients or couples at risk.

The proteolytic disease peptides for Hb E genotype have been identified to include the mutation of amino acid 26, thus including peptides having any portion of [SEQ ID NO. 5] that encompasses residue 26.

Thus, in the exemplified embodiment, the disease or disorder to be detected may be a hemoglobinopathy, including for example α-thalassemia, β-thalassemia, sickle cell anemia or hemolyticanemia. In one embodiment, the disease is α-thalassemia. In one embodiment, the disease is β-thalassemia.

The sample may be a peripheral blood sample containing red blood cells from an individual.

The disease allele may be the α-hemoglobin gene or the β-hemoglobin gene. For example, the disease allele may be an α-hemoglobin gene containing the Hb CS, Hb PS, Hb Queens, Hb J-Buda or Hb Q-Thailand mutation. For example, the disease allele may be a β-hemoglobin gene containing the Hb E, Hb S, Hb C, Hb D-Punjab or Hb O-Arab mutation.

In some embodiments, the disease or disorder is α-thalassemia.

In some embodiments, the proteolytic peptide may consist of one of the following sequences:

[SEQ ID NO. 6]
VHLTPEEKSAVTALW,
[SEQ ID NO. 7]
TALWGKVNVDEVGGEALG,
[SEQ ID NO. 8]
ALWGKVNVDEVGGEALG,
[SEQ ID NO. 9]
WGKVNVDEVGGEAL,
[SEQ ID NO. 10]
GKVNVDEVGGEAL,
[SEQ ID NO. 11]
VDEVGGEALGRLLV,
[SEQ ID NO. 12]
LVVYPWTQRF,
[SEQ ID NO. 13]
RFFESFGDLSTPDAV,
[SEQ ID NO. 14]
FFESFGDLSTPDAV,
[SEQ ID NO. 15]
AFSDGLAHLDNLKGTFAT,
[SEQ ID NO. 16]
AFSDGLAHLDNLKGTFA,
[SEQ ID NO. 17]
SDGLAHLDNLKGTF,
[SEQ ID NO. 18]
DGLAHLDNLKGTFA,
[SEQ ID NO. 19]
DGLAHLDNLKGTFATL,
[SEQ ID NO. 20]
YQKVVAGVANALAHKYH
or
[SEQ ID NO. 21]
VAGVANALAHKYH,
[SEQ ID NO. 22]
GASVAVPPARWASQ,
[SEQ ID NO. 23]
GASVAVPPARWAS,
[SEQ ID NO. 24]
GASVAVPPARWA,
[SEQ ID NO. 25]
GASVAVPPARW,
[SEQ ID NO. 26]
ASVAVPPARWASQ,
[SEQ ID NO. 27]
SVAVPPARWASQRALLPS,
[SEQ ID NO. 28]
SVAVPPARWASQ,
[SEQ ID NO. 29]
SVAVPPARWAS,
[SEQ ID NO. 30]
SVAVPPARW,
[SEQ ID NO. 31]
VAVPPARWASQ,
[SEQ ID NO. 32]
AVPPARWASQRALLPSLHRPFLVFE,
[SEQ ID NO. 33]
AVPPARWASQRALLPSL,
[SEQ ID NO. 34]
AVPPARWASQRALLPS,
[SEQ ID NO. 35]
VPPARWASQRALLPSL,
[SEQ ID NO. 36]
VPPARWASQRALLPS,
[SEQ ID NO. 37]
VPPARWASQR,
[SEQ ID NO. 38]
VPPARWASQ,
[SEQ ID NO. 39]
VPPARWAS,
[SEQ ID NO. 40]
ASQRALLPSLHRPFLVFE,
[SEQ ID NO. 41]
ASQRALLPSLHRPFL,
[SEQ ID NO. 42]
ASQRALLPSLHRPF,
[SEQ ID NO. 43]
SQRALLPSLHRPFLVFE,
[SEQ ID NO. 44]
SQRALLPSLHRPFL,
[SEQ ID NO. 45]
SQRALLPSLHRPF,
[SEQ ID NO. 46]
SQRALLPSLHRP,
[SEQ ID NO. 47]
SQRALLPSL,
[SEQ ID NO. 48]
QRALLPSLHRPFLVFE,
[SEQ ID NO. 49]
QRALLPSLHRPFL,
[SEQ ID NO. 50]
QRALLPSLHRPF,
[SEQ ID NO. 51]
QRALLPSLHRP,
[SEQ ID NO. 52]
RALLPSLHRPFLVFE,
[SEQ ID NO. 53]
RALLPSLHRPFL,
[SEQ ID NO. 54]
RALLPSLHRPF,
[SEQ ID NO. 55]
RALLPSLHRP,
[SEQ ID NO. 56]
ALLPSLHRPFLVFE,
[SEQ ID NO. 57]
ALLPSLHRPFL,
[SEQ ID NO. 58]
ALLPSLHRPF;
[SEQ ID NO. 59]
ALLPSLHRP,
[SEQ ID NO. 60]
LLPSLHRPFLVFE,
[SEQ ID NO. 61]
LLPSLHRPFLVF,
[SEQ ID NO. 62]
LLPSLHRPF,
[SEQ ID NO. 63]
LPSLHRPFLVFE,
[SEQ ID NO. 64]
LPSLHRPFLVF,
[SEQ ID NO. 65]
LPSLHRPFLV,
[SEQ ID NO. 66]
LPSLHRPFL,
[SEQ ID NO. 67]
LPSLHRPF,
[SEQ ID NO. 68]
LPSLHRP,
[SEQ ID NO. 69]
PSLHRPFLVFE,
[SEQ ID NO. 70]
PSLHRPF,
[SEQ ID NO. 71]
SLHRPFLVFE,
[SEQ ID NO. 72]
LHRPFLVFE,
[SEQ ID NO. 73]
HRPFLVFE,
[SEQ ID NO. 74]
HRPFLVF,
[SEQ ID NO. 75]
RPFLVFE,
[SEQ ID NO. 76]
SVAVPPARWASQRALLPSLHRPFLVFE,
[SEQ ID NO. 77]
PARWASQR,
or
[SEQ ID NO. 78]
ALLPSLHR.

In some embodiments, the disease or disorder is β-thalassemia.

In some embodiments, the proteolytic peptide may consist of one of the following sequences:

[SEQ ID NO. 79]
VLSPADKTNV,
[SEQ ID NO. 80]
VLSPADKTNVKAAWGKV,
[SEQ ID NO. 81]
AAWGKVGAHAGEYGAEALE,
[SEQ ID NO. 82]
GKVGAHAGEYGAEALERM,
[SEQ ID NO. 83]
AHAGEYGAEALE,
[SEQ ID NO. 84]
TYFPHFDLSHGSAQV,
[SEQ ID NO. 85]
ALTNAVAHVDDMPN,
[SEQ ID NO. 86]
VDDMPNALSAL,
[SEQ ID NO. 87]
TLAAHLPAEFTPAVH,
[SEQ ID NO. 88]
LAAHLPAEFTPAVH,
[SEQ ID NO. 89]
SLDKFLASVSTVLTSKYR,
[SEQ ID NO. 90]
DKFLASVSTVLTSKYR,
[SEQ ID NO. 91]
KFLASVSTVLTSKYR
or
[SEQ ID NO. 92]
ASVSTVLTSKYR,
[SEQ ID NO. 93]
VHLTPEEKSAVTALWGKVNVDEVGGKALGRLLVVYPWTQRF,
[SEQ ID NO. 94]
VHLTPEEKSAVTALWGKVNVDEVGGKALGRLLVVYPWTQ,
[SEQ ID NO. 95]
VHLTPEEKSAVTALWGKVNVDEVGGKALGRLLVV,
[SEQ ID NO. 96]
VHLTPEEKSAVTALWGKVNVDEVGGKALGRLLV,
[SEQ ID NO. 97]
VHLTPEEKSAVTALWGKVNVDEVGGKALGRLL,
[SEQ ID NO. 98]
VHLTPEEKSAVTALWGKVNVDEVGGKALGRL,
[SEQ ID NO. 99]
VHLTPEEKSAVTALWGKVNVDEVGGKALG,
[SEQ ID NO. 100]
VHLTPEEKSAVTALWGKVNVDEVGGKA,
[SEQ ID NO. 101]
LTPEEKSAVTALWGKVNVDEVGGKALG,
[SEQ ID NO. 102]
TPEEKSAVTALWGKVNVDEVGGKALGRLLV,
[SEQ ID NO. 103]
KSAVTALWGKVNVDEVGGKALGR,
[SEQ ID NO. 104]
KSAVTALWGKVNVDEVGGKALG,
[SEQ ID NO. 105]
SAVTALWGKVNVDEVGGKALGRLLV,
[SEQ ID NO. 106]
SAVTALWGKVNVDEVGGKALG,
[SEQ ID NO. 107]
TALWGKVNVDEVGGKALGRLLVVYPWTQRF,
[SEQ ID NO. 108]
TALWGKVNVDEVGGKALGRLLVVYPWTQ,
[SEQ ID NO. 109]
TALWGKVNVDEVGGKALGRLLVVYP,
[SEQ ID NO. 110]
TALWGKVNVDEVGGKALGRLLVV,
[SEQ ID NO. 111]
TALWGKVNVDEVGGKALGRLLV,
[SEQ ID NO. 112]
TALWGKVNVDEVGGKALGRLL,
[SEQ ID NO. 113]
TALWGKVNVDEVGGKALGRL,
[SEQ ID NO. 114]
TALWGKVNVDEVGGKALGR,
[SEQ ID NO. 115]
TALWGKVNVDEVGGKALG,
[SEQ ID NO. 116]
TALWGKVNVDEVGGKAL,
[SEQ ID NO. 117]
TALWGKVNVDEVGGKA,
[SEQ ID NO. 118]
TALWGKVNVDEVGGK,
[SEQ ID NO. 119]
ALWGKVNVDEVGGKALGRLLV,
[SEQ ID NO. 120]
ALWGKVNVDEVGGKALGRLL,
[SEQ ID NO. 121]
ALWGKVNVDEVGGKALGR,
[SEQ ID NO. 122]
ALWGKVNVDEVGGKALG,
[SEQ ID NO. 123]
ALWGKVNVDEVGGKAL,
[SEQ ID NO. 124]
ALWGKVNVDEVGGKA,
[SEQ ID NO. 125]
ALWGKVNVDEVGGK,
[SEQ ID NO. 126]
LWGKVNVDEVGGKALGRLLVVYPWTQRF,
[SEQ ID NO. 127]
LWGKVNVDEVGGKALGRLLVVYPWTQ,
[SEQ ID NO. 128]
LWGKVNVDEVGGKALGRLLVVYP,
[SEQ ID NO. 129]
LWGKVNVDEVGGKALGRLLV,
[SEQ ID NO. 130]
LWGKVNVDEVGGKALGRL,
[SEQ ID NO. 131]
LWGKVNVDEVGGKALGR,
[SEQ ID NO. 132]
LWGKVNVDEVGGKALG,
[SEQ ID NO. 133]
LWGKVNVDEVGGKAL,
[SEQ ID NO. 134]
LWGKVNVDEVGGK,
[SEQ ID NO. 135]
WGKVNVDEVGGKALGRLLVVYPWTQ,
[SEQ ID NO. 136]
WGKVNVDEVGGKALGRLLVVYP,
[SEQ ID NO. 137]
WGKVNVDEVGGKALGRLLV,
[SEQ ID NO. 138]
WGKVNVDEVGGKALGRLL,
[SEQ ID NO. 139]
WGKVNVDEVGGKALGRL,
[SEQ ID NO. 140]
WGKVNVDEVGGKALGR,
[SEQ ID NO. 141]
WGKVNVDEVGGKALG,
[SEQ ID NO. 142]
WGKVNVDEVGGKAL,
[SEQ ID NO. 143]
WGKVNVDEVGGK,
[SEQ ID NO. 144]
GKVNVDEVGGKALGRLLVVYPWTQ,
[SEQ ID NO. 145]
GKVNVDEVGGKALGRLLVVYP,
[SEQ ID NO. 146]
GKVNVDEVGGKALGRLLV,
[SEQ ID NO. 147]
GKVNVDEVGGKALGRLL,
[SEQ ID NO. 148]
GKVNVDEVGGKALGRL,
[SEQ ID NO. 149]
GKVNVDEVGGKALGR,
[SEQ ID NO. 150]
GKVNVDEVGGKALG,
[SEQ ID NO. 151]
GKVNVDEVGGKAL,
[SEQ ID NO. 152]
GKVNVDEVGGKA,
[SEQ ID NO. 153]
GKVNVDEVGGK,
[SEQ ID NO. 154]
KVNVDEVGGKALGRLLV,
[SEQ ID NO. 155]
KVNVDEVGGKALGRLL,
[SEQ ID NO. 156]
KVNVDEVGGKALGRL,
[SEQ ID NO. 157]
KVNVDEVGGKALGR,
[SEQ ID NO. 158]
KVNVDEVGGKALG,
[SEQ ID NO. 159]
KVNVDEVGGKAL,
[SEQ ID NO. 160]
KVNVDEVGGKA,
[SEQ ID NO. 161]
KVNVDEVGGK,
[SEQ ID NO. 162]
VNVDEVGGKALGRLLVVYPWTQ,
[SEQ ID NO. 163]
VNVDEVGGKALGRLLVVYP,
[SEQ ID NO. 164]
VNVDEVGGKALGRLLV,
[SEQ ID NO. 165]
VNVDEVGGKALGRL,
[SEQ ID NO. 166]
VNVDEVGGKALGR,
[SEQ ID NO. 167]
VNVDEVGGKALG,
[SEQ ID NO. 168]
VNVDEVGGKAL,
[SEQ ID NO. 169]
VNVDEVGGKA,
[SEQ ID NO. 170]
VNVDEVGGK,
[SEQ ID NO. 171]
NVDEVGGKALGRLLVVYPWTQRF,
[SEQ ID NO. 172]
NVDEVGGKALGRLLVVYPWTQ,
[SEQ ID NO. 173]
NVDEVGGKALGRLLV,
[SEQ ID NO. 174]
NVDEVGGKALGRLL,
[SEQ ID NO. 175]
NVDEVGGKALGR,
[SEQ ID NO. 176]
NVDEVGGKALG,
[SEQ ID NO. 177]
NVDEVGGKAL,
[SEQ ID NO. 178]
VDEVGGKALGRLLVVYPWTQRF,
[SEQ ID NO. 179]
VDEVGGKALGRLLVVYPWTQ,
[SEQ ID NO. 180]
VDEVGGKALGRLLV,
[SEQ ID NO. 181]
VDEVGGKALGRLL,
[SEQ ID NO. 182]
VDEVGGKALGR,
[SEQ ID NO. 183]
VDEVGGKALG,
[SEQ ID NO. 184]
VDEVGGKAL,
[SEQ ID NO. 185]
VDEVGGKA,
[SEQ ID NO. 186]
DEVGGKALGRLLVVYPWTQRF,
[SEQ ID NO. 187]
DEVGGKALGRLLVVYPWTQ,
[SEQ ID NO. 188]
DEVGGKALGRLLV,
[SEQ ID NO. 189]
DEVGGKALGRLL,
[SEQ ID NO. 190]
DEVGGKALGR,
[SEQ ID NO. 191]
EVGGKALGRLLVVYPWTQRF,
[SEQ ID NO. 192]
EVGGKALGRLLVVYPWTQ,
[SEQ ID NO. 193]
EVGGKALGRLLV,
[SEQ ID NO. 194]
EVGGKALGRLL,
[SEQ ID NO. 195]
EVGGKALGR,
[SEQ ID NO. 196]
VGGKALG,
[SEQ ID NO. 197]
GGKALGRLLVVYPWTQRF,
[SEQ ID NO. 198]
GGKALGRLLVVYPWTQ,
[SEQ ID NO. 199]
GGKALGRLLVVY,
[SEQ ID NO. 200]
GGKALGRLLV,
or
[SEQ ID NO. 201]
GGKALGRLL.

Where the disease allele is an α-hemoglobin gene containing the Hb CS or Hb PS mutation, the proteolytic disease peptide that is used as a biomarker may be any peptide consisting of one of the peptide sequences set out in Table 3.

Where the disease allele is a β-hemoglobin gene containing the Hb E mutation, the disease proteolytic peptide that is used as a biomarker may be any peptide consisting of one of the peptide sequences set out in Table 4.

The present methods and uses are further exemplified by way of the following non-limiting examples.

### EXAMPLES

### Example 1

As an example disease, the method was tested in α-thalassemia patients with stop-codon mutations. The mutations result in elongated α-globin that is longer than the α-globin found in a healthy individual not carrying a disease allele, and thus would result in unique, proteolytic peptides specific to the mutant disease allele. In the results set out below, several peptides were identified that matched with the C-terminal fragments of the elongated α-globin, as predicted. The mutations identified are those that have been identified in peripheral blood carrying the Hb CS (Hb Constant Spring) or PS (Pakse) allele, including heterozygotes and CS double heterozygotes with HbE.

Materials and Methods

Blood sample collection: Peripheral blood samples in EDTA were collected from Nalchonpathom hospital of Mahidol University in Thailand, and KK Women's and Children's Hospital (KKH) in Singapore. Ethical clearances including informed consent were approved by the Institutional Review Board of Mahidol University, KKH and Nanyang Technological University. Whole blood, washed (with normal saline) and unwashed packed red cells were processed between 6 hrs and a few days in various conditions with and without adding a cocktail of protease inhibitors. All blood samples were stored at −80° C.

MALDI-TOFMS analysis: One microliter of whole blood was diluted to 500 μl with 50% acetonitrile/0.1% trifluoroacetic acid. About 0.3 μl of the diluted blood was spotted onto a MALDI target, immediately mixed with an equal volume of sinapinic acid (MALDI grade, Sigma-Aldrich, Steinheim, Switzerland) solution (8 mg/ml in 50% acetonitrile/0.1% trifluoroacetic acid) and let dry in ambient temperature. The crystallized sample was introduced into a MALDI-TOF/TOF mass spectrometer (4800, Applied Biosystems, California, USA). Intact globin chains were detected using the linear mode of the TOF/TOF, which was calibrated using a mixture of standard proteins in a neighbouring spot.

Peptide extraction for MALDI: Tris powder (Sigma-Aldrich, Steinem, Switzerland), hydrochloric acid (Merck, Darmstadt, Germany) and cocktail protease inhibitors (Roche Diagnostics GmbH, Roche Applied Science, Mannheim, Germany) were used to prepare 50 mMTris-HCl buffer in 18.2 MΩ milliQ water, pH 7.8. Twenty microliters of frozen blood were dissolved and diluted 10 times in the Tris-HCl buffer. Diluted protein was further denatured in equal volume of 50% acetonitrile with 0.2% formic acid (Merk, Damstadt, Germany). To remove cell debris, hemolysate was spun down with 10,000 g for 7 minutes and only the supernatant was collected. The supernatant containing peptides and proteins was further separated using a 10 kDa molecular weight cut-off (MWCO) centrifugal filter device (Microcon, Millipore Corporation, Bedford, USA). The eluent that passed through the Microcon spin column was collected and peptides were concentrated and desalted using a reverse-phase C18 ZipTip (Millipore Corporation, Billerica, USA) and eluted with 2.5 μl of 70% acetonitrile and 0.1% TFA.

MALDI-MS and MS/MS analysis for peptide identification: Purified peptides (0.3 μl) were spotted onto the MALDI plate, followed by an equal volume of 8 mg/mL α-cyano-4-hydroxycinnaamic acid (MALDI grade, Sigma-Aldrich, Steinheim, Switzerland) matrix dissolved in 50% acetronitile/0.1% trifluoroacetic acid. The plate was let dry in ambient environment and the crystallized samples were analyzed in a MALDI-TOF/TOF mass spectrometer (4800, Applied Biosystems, California, USA) using reflector mode for positive ions. MS spectra were generated and precursor ions were chosen to be fragmented in tandem MS by using collision-induced dissociation (CID). After obtaining tandem mass spectra, a de novo sequencing software, DeNovo Explorer (Applied Biosystems), was used to generate a list of candidate peptide sequences, which were automatically submitted for protein identification using ProBLAST database search.

Peptide extraction for LC/MS: 50 mM Tris-HCl buffer in the presence of cocktail protease inhibitors, pH 7.8 (Roche Diagnostics GmbH, Mannheim, Germany), was used to lyse red blood cells at a 1:1 proportion. 200 microliters of hemolysate was further diluted 2 times in the 50% acetonitrile and 0.2% formic acid buffer (Merk, Darmstadt, Gennany). Diluted blood was then spun down in order to remove cell debris, with 10,000 g for 5 minutes and the supernatant was filtrated using the Microcon 10 kDa molecular weight cut-off centrifugal filter devices. The eluent that passed through the spin column was collected and peptides were then concentrated and desalted using a reverse-phase C18 ZipTip and eluted with 5 μl of 70% acetonitrile and 0.1% trifluoroacetic acid. Purified peptides were dried using a speed-vacuum concentrator (Eppendorf 5301, Hamburg, Germany) and reconstituted in 20 μl of 2% acetonitrile and 0.1% formic acid before submitting to LC-MS/MS and MRM-LC/MS.

LC/MS and MS/MS: A nano LC system (LC Packings, Amsterdam, The Netherlands) was used for LC/MS. Purified peptide samples were injected onto a reverse phase column using an autosampler. The injection volume was 1 μl . A nano column (Integrafrit, New Objective, Woburn Mass., USA, 75 μm ID, 360 μm OD, 10 cm) self-packed with 4 μm C12 reverse phase particles (Jupiter Proteo, Phenomenex, Torrance, Calif., USA) was used for LC separation. A 50-min gradient with mobile phases A (2% acetonitrile, 0.1% formic acid) and B (98% acetonitrile, 0.1% formic acid) at a flow rate of 300 nl/min was used to elute peptides. The MS and MS/MS spectra were acquired with an LTQ-Orbitrap (Thermo, Bremen, Germany). The results were searched with UniProt human protein database with added Hb CS and Hb PS sequences using the Sequest search engine (Bioworks 3.3, Thermo). Key search parameters were: enzyme specificity=none, MS accuracy=20 ppm, MS/MS accuracy=0.5 Da. Post-search filtering was used with the following setting: different peptides, with peptide probability >0.5.

Multiple reaction monitoring (MRM): Selected peptide precursors and fragments were monitored using an LTQ linear ion trap. SRM mode was selected for precursor/fragment transition monitoring, with mass window of 1 Da m/z (±0.5 Da). LC gradient was 3.2% acetonitrile from 0 to 5 minutes, 3.2% acetonitrile to 32% acetonitrile from 5 to 15 minutes, followed by washing at 90% acetonitrile for 10 minutes and re-equilibration of 3.2% acetonitrile for 15 minutes. The flow rate was 4 μl/min, using Dionex3000 RSLC with a 5 μm C18 column (Michrom Biosciences, 0.1 mm×15 mm) and a Michrom Captive Spray ESI source.

Results

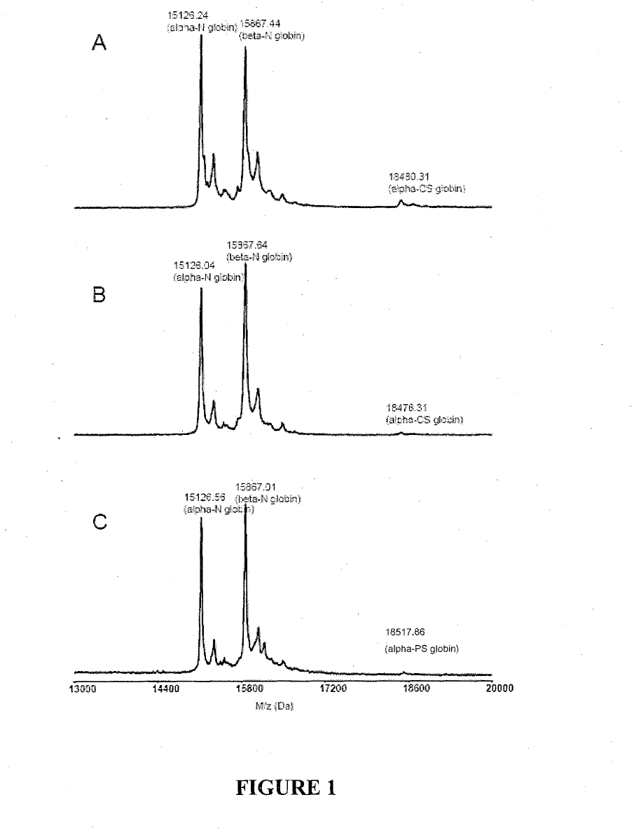

Determination of minor globin molecular weight: Detection of the minor elongated α-globin chain by direct MALDI-TOF measurement of diluted blood samples can be achieved in homozygous Hb CS (FIG. 1A), Hb H-CS (FIG. 1B), and Hb H-PS (FIG. 1C) with molecular mass around 18481 kDa for α^{CS} and 18516 Da for α^{PS}, respectively. Homozygous Hb PS was not tested as this genotype was not available. All samples tested by MALDI are listed in Table 1. In heterozygous Hb CS, the elongated α-globin chain was marginally detected. This is expected as heterozygotes for this mutation have only ˜1% Hb CS in their erythrocytes.

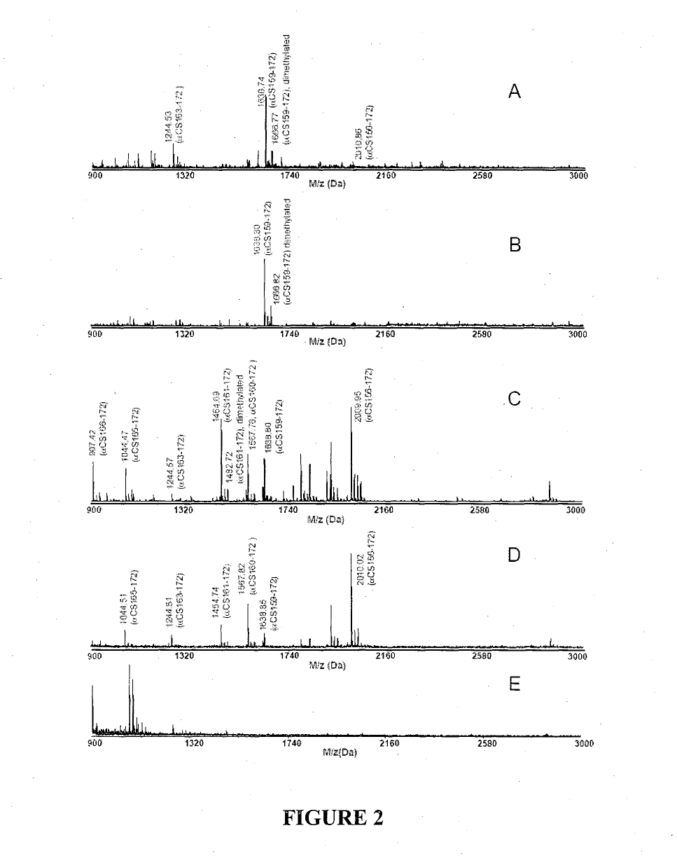

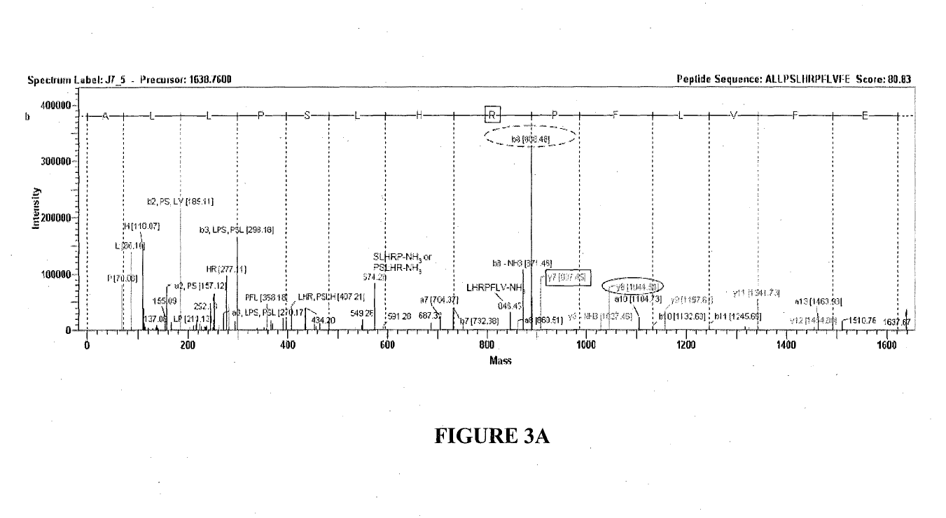

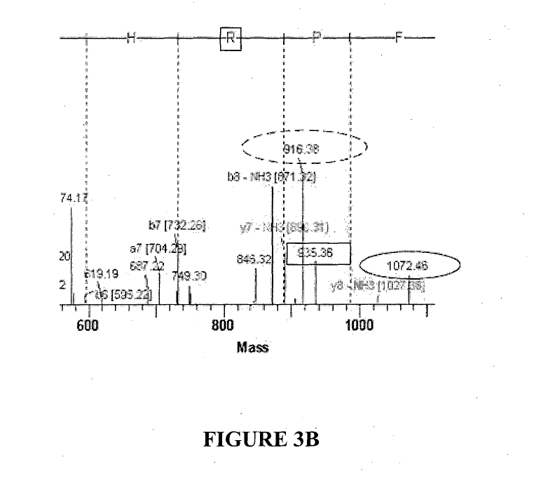

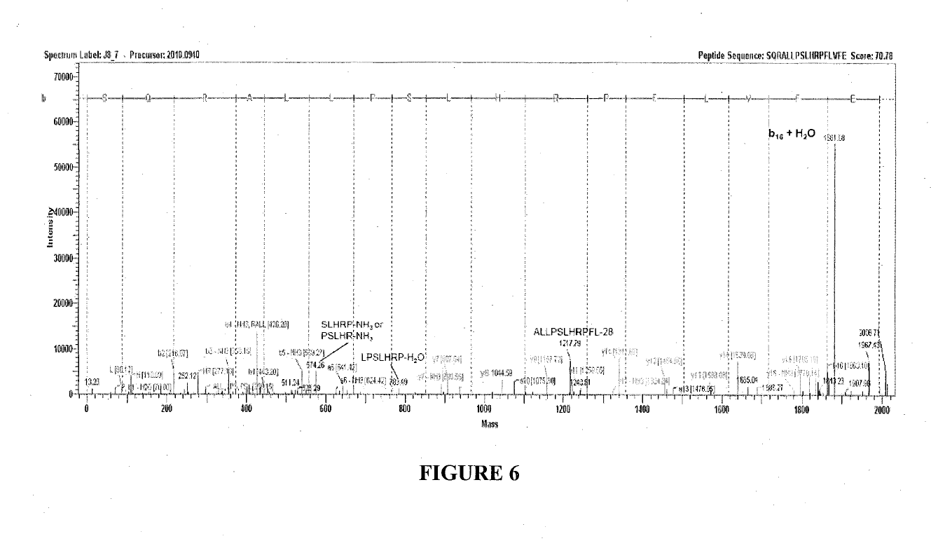

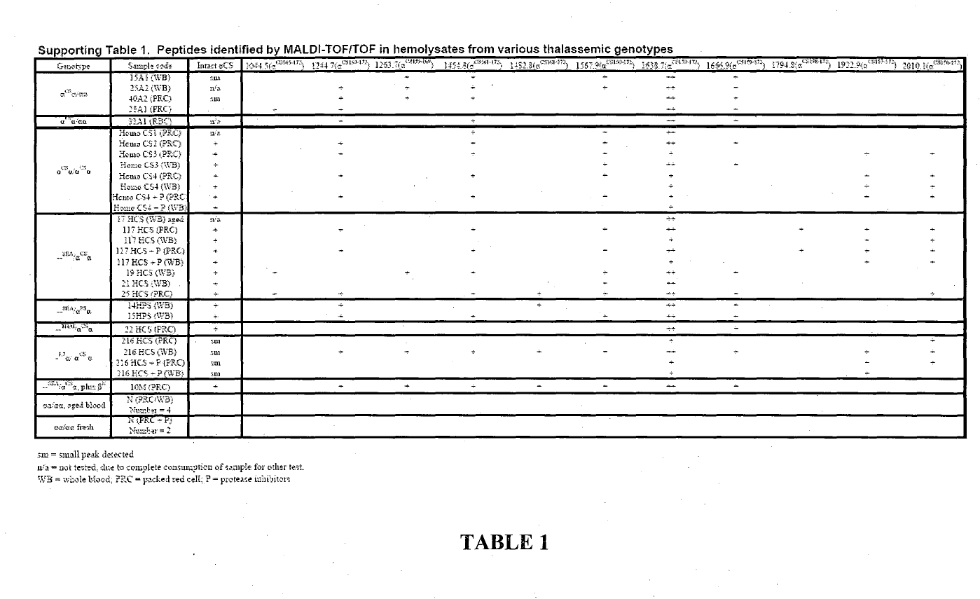

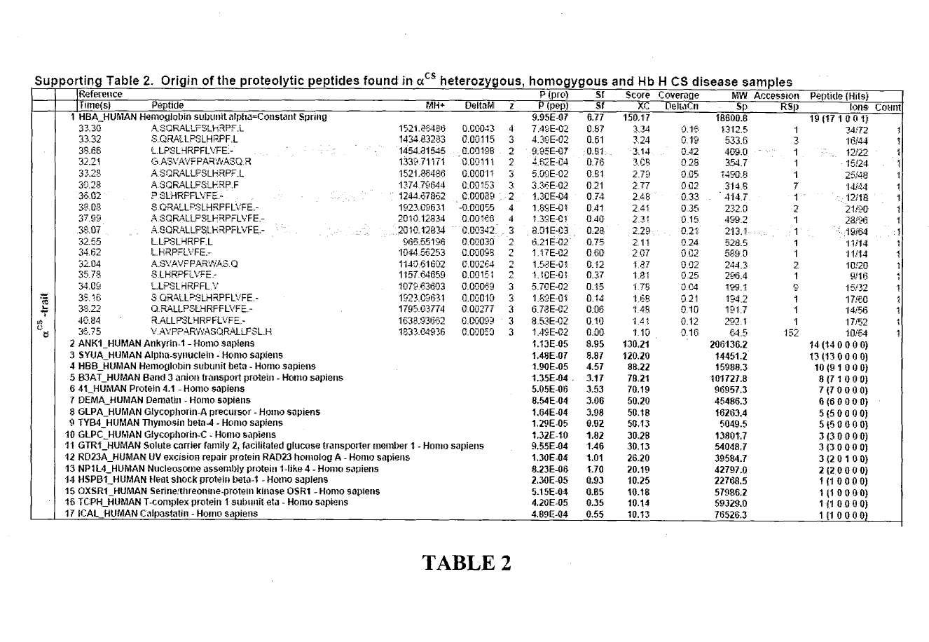

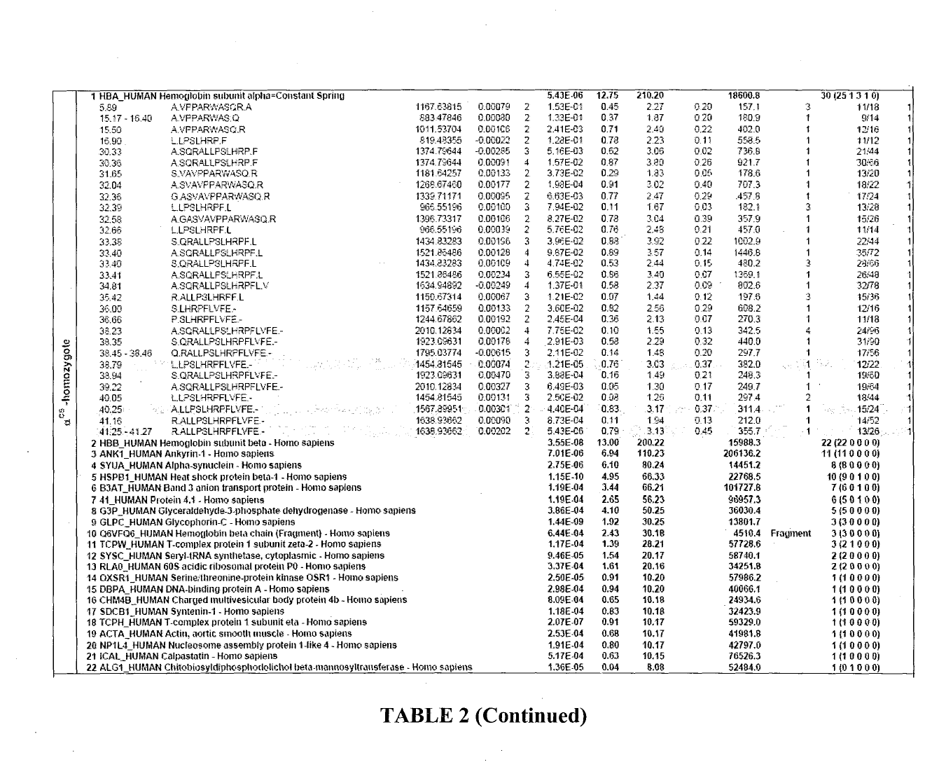

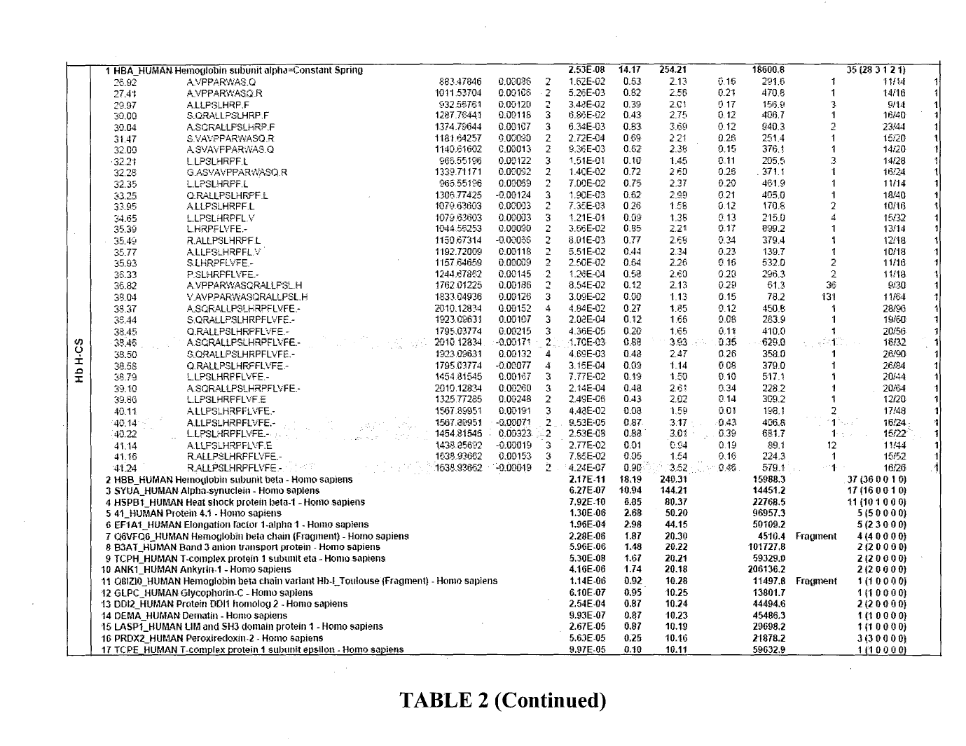

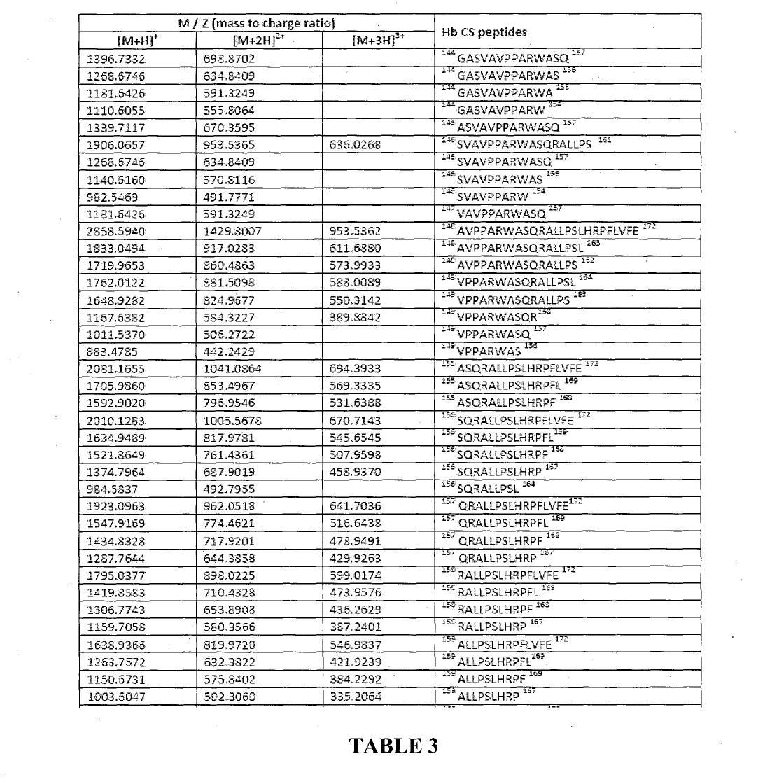

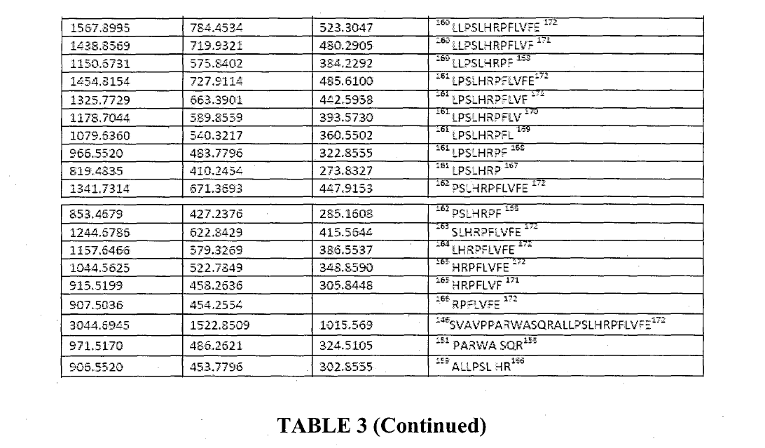

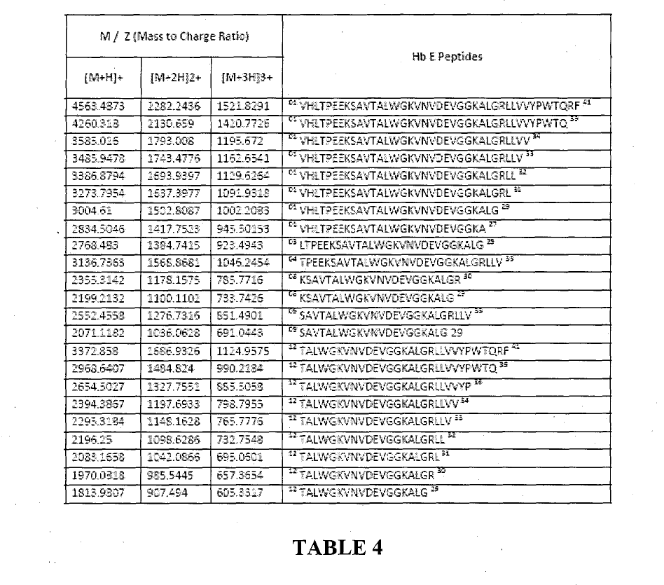

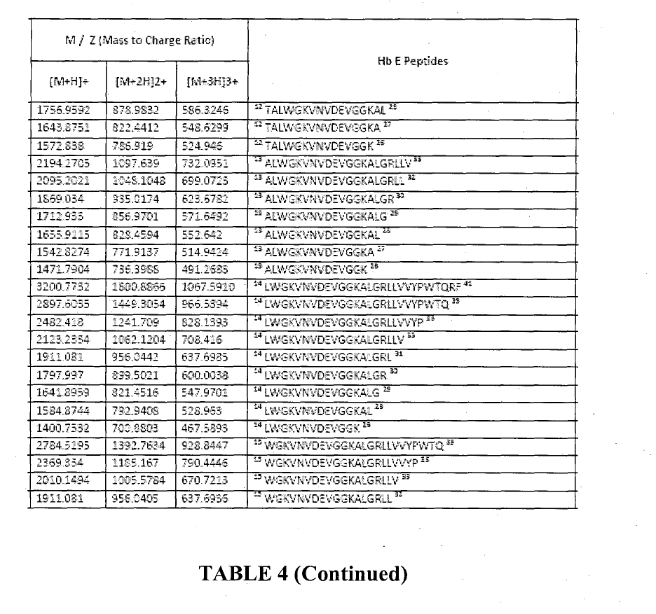

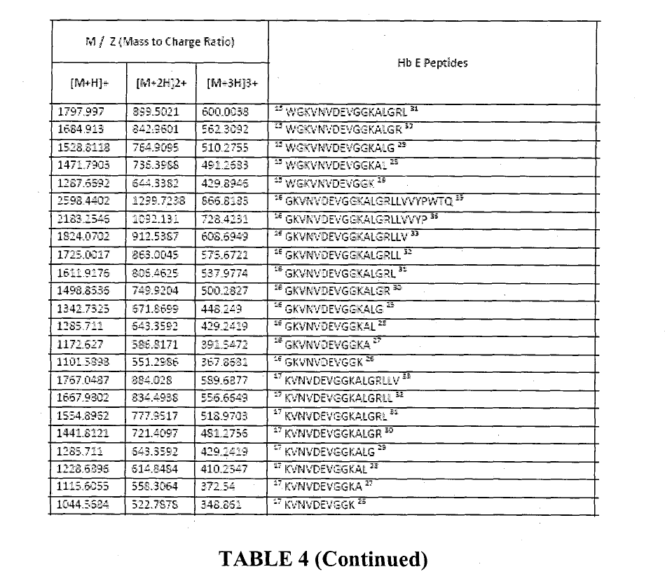

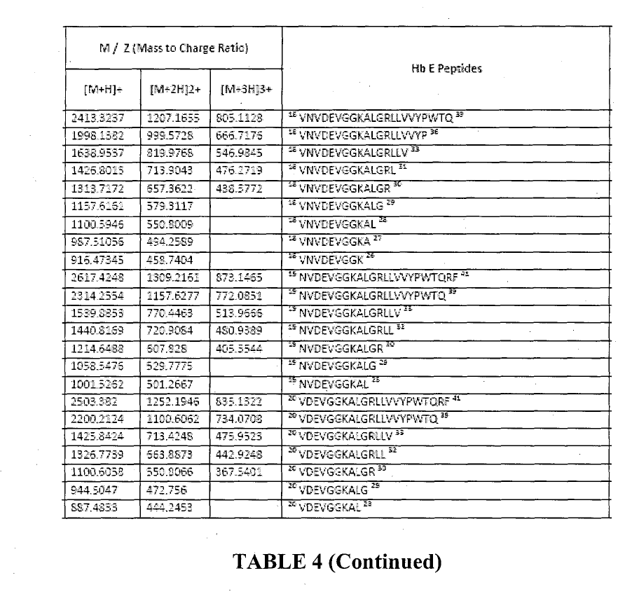

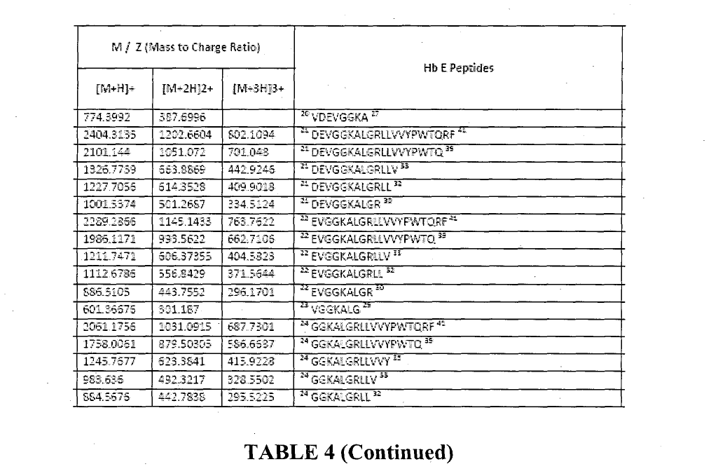

MALDI-TOF MS/MS for identification of selected peptide sequences: In order to find biomarkers that are specific to disease genotypes, we tried to look for proteolytic peptides in red blood cells. Intact proteins were removed by using a 10-kDa molecular weight cut-off (MWCO) membrane. The filtrate containing peptides was desalted and concentrated with a C18 ZipTip and then subject to MALDI-TOFMS analysis. Normal donor samples (data not shown) were tested as controls but few peptides could be found and they did not match with the masses from α^{CS} protein fragments. In patient samples with abnormal hemoglobin variant of Hb CS or PS, peptides were consistently observed with [M+H]⁺ values of 1638.7 Da or 2010.1 Da among others (FIG. 2 and Table 1). The 2010.1 Da peak was usually observed in freshly collected samples, mostly with added protease inhibitors. The sequences of these peptides were identified in MS/MS spectra and matched to the C-terminal end of the elongated α-globin chain α^{CS159-172} and α^{CS156-172}, respectively (FIG. 3A and FIG. 6). The dominant fragment (1881.08 Da) in the MS/MS spectra of 2010.10 Da (FIG. 6) was identified to be b₁₆+H₂O. Such type of CID fragmentation (bₙ₋₁+H₂O) is often observed for peptides with a basic amino acid residual at any non-C-terminal position [25].

Identification of modified peptides: The elongated part of α^{CS} globin was suspected to be partially modified since two fragments showed the same sequence with different m/z values 1638.74 Da→4 1666.77 Da and 1454.60 Da→1482.67 Da, respectively. As shown in FIG. 3B, an addition of 28 Da could be detected on arginine at amino acid position 166 (¹⁵⁹ALLPSLHRPFLVFE [SEQ ID NO. 56]). This modification could be dimethylation of arginine. A few other peptides identified also include this position although not all of them were methylated (1044.5 Da,α^{CS165-172}, 1567.7 Da,α^{CS160-172}; and 2010.1 Da,α^{CS156-172}).

Summary of samples tested by MALDI-MS: All samples tested are listed in Table 1. Signature peptides of α^{CS} degradation products were identified with MS/MS as follows: 907.4 Da (α^{CS166-172}), 1044.5 Da (α^{CS165-172}), 1244.6 Da (α^{CS163-172}), 1263.7 Da (α^{CS159-169}), 1454.7 Da (α^{CS161-172}), 1482.7 Da (α^{CS161-172}dimethylated),1567.8 Da (α^{CS160-172}), 1638.7 Da (α^{CS159-172}), 1666.9 Da (α^{CS159-172} dimethylated), 1794.8 Da (α^{CS158-172}), 1922.9 Da (α^{CS157-172}) and 2010.1 Da (α^{CS156-172}). Similar fragments were also found in α^{PS} samples. As the only amino acid difference between α^{CS} and α^{PS} is at α142 (Q142Y), proteolytic fragments not including this particular position are identical for both α^{CS} and α^{PS}.

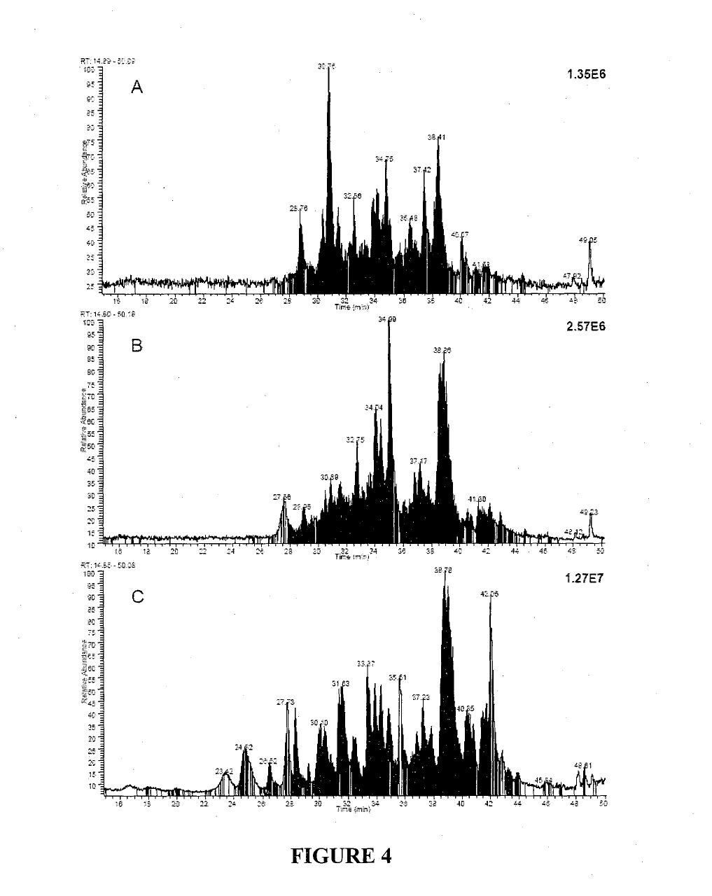

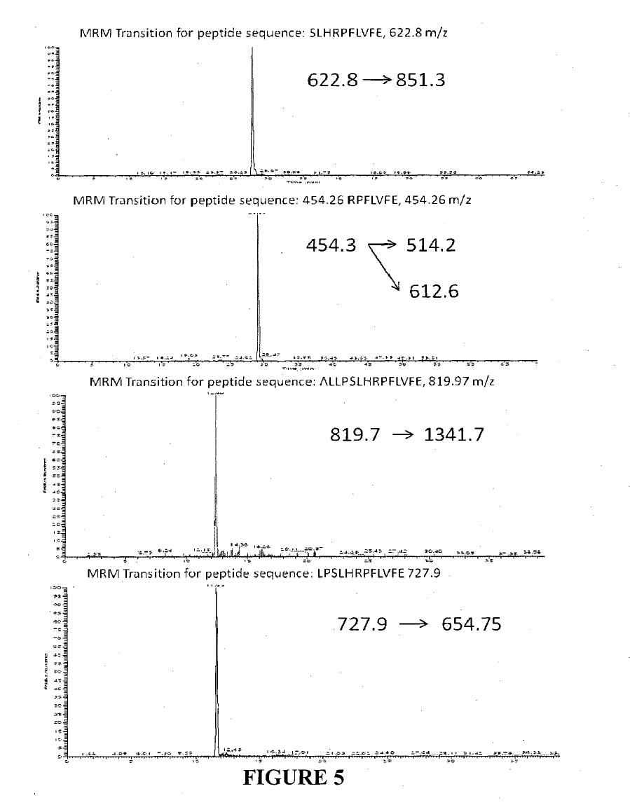

Identification of proteolytic peptides with LC/MS/MS: In order to identify more proteolytic peptides and possibly peptides from proteins other than hemoglobin, nano-flow LC/MS was used to separate the purified peptide mixture. Representative total ion chromatograms are shown in FIG. 4 for α^{CS} heterozygote, homozygote and Hb H CS genotypes respectively. These results indicate that increasing peptide fragments were detected in homozygous and Hb H CS samples respectively when compared with CS trait samples. This may represent the severity of the disease. Corresponding database search results are listed in Table 2. As expected, proteolytic peptides from the elongated a globin chain contributed to all the peptides used to identify the Hb CS protein. No peptides from other parts of the α globin were detected, indicating that the proteases in the red blood cells were targeting the elongated portion of the Hb CS specifically. Hemoglobin β chain fragments were also detected extensively and their sequences matched to different parts of the β globin chain. This observation supports the assumption that in α-thalassemia, excess β globin chain are degraded preferentially as a body's response to the disease by attempting to restore the 1:1 ratio of α/β. Interestingly, degradation of heat shock protein beta-1 and glycophorin-C was exclusively at the C-terminal end in all three genotypes, similarly to that of the Hb CS. Other fragments observed came from erythrocyte proteins that may related to the disease in someway. Proteolytic peptides carrying partial Hb CS sequences were detected using LC/MS from 14 different patient samples carrying the Hb CS allele. The results were filtered with the Trans Proteomic Pipeline with Peptide Prophet at the 5% false discovery level and summarized in Table 3.

Multiple reactionmonitoring of α^{CS} proteolytic peptides: From the LC/MSMS data obtained above, selected precursor-fragment pairs were chosen for MRM using a linear ion trap mass spectrometer running at high LC flow rate. Representative spectra are shown in FIG. 5. It is a proof-of-principle demonstration that these specific precursor-fragment pairs can be monitored in a high throughput fashion, similar to neonatal metabolic screening. And it can be used for large scale validation of these selected candidate markers.

Discussion

The common forms of α-thalassemia in Southeast Asia region are Hb H disease and Hb Bart's hydropsfetalis. The affected children who are compound heterozygous for non-deletional mutations, especially Hb CS and Hb PS, and α-thalassemia 1 (2-gene deletion) usually result in a more severe phenotype than the classical Hb H disease due to 3 gene deletion (α-thalassemia 1/α-thalassemia 2). Furthermore, Bart's hydropsfetalis syndrome can occur even in homozygosity of Hb CS [26]. It is therefore very desirable for diagnostic laboratories to have a simple and standardized test for high-throughput screening. Traditionally, when Hb stability (unstable Hb variant, unstable Hb tetramer) is suspected from clinical symptoms, special tests such as Heinz body preparation, heat stability and erythrocyte inclusion bodies staining can be used for screening. However, these procedures are less sensitive and more labour-intensive, leading to misdiagnosis [27, 28]. On the other hand, a validated biomarker specific for certain disease alleles hold promise for high-throughput screening before selected individuals are identified for detailed DNA sequence analysis.

Proteolytic peptides identified in this study have the potential to serve as valuable biomarkers. Since these peptides originated from the elongated portion of the α-globin chain, they are specific for stop codon mutations. Such biomarkers can be screened the same way as metabolic profiling for newborns using selected reaction monitoring (SRM). The proteolytic peptide makers identified in this study do not distinguish between Hb CS and Hb PS since the peptide sequences do not cover the stop codon position. To confirm the genotype, DNA analysis can be performed. Direct measurement of protein masses by MS provides another alternative. The mass accuracy of simple MALDI-MS in linear TOF mode is sufficient to distinguish between Hb CS (UAA142CAA, Gln) and Hb PS (UAA142UAU/UAC, Tyr). Other types of chain-termination mutations such as Hb Koya Dora (UAA142UCA, Ser) and the predicted one (UAA142UAU, Leu), can also be uniquely identified by direct mass measurement. The remaining two mutations, Hb Icaria(UAA142AAA, Lys) and Hb Seal Rock (UAA142GAA, Glu) may be differentiated from Hb CS by high resolution ESI-FTICR-MS with electron capture dissociation (ECD) capability, or by an endoproteinase (such as trypsin, Lys-C or Glu-C) digest followed by peptide mass fingerprinting.

The same signature peptides may be observed in other more complex genotypes, such as Hb CS heterozygote combined with P-thalassemia, which usually interacts with the common β-chain variant Hb E. It should be pointed out that misdiagnosis of Hb CS in individuals with Hb E can happen in routine hematological analysis. Some of the double heterozygous Hb CS/Hb E was detected by DNA analysis only because the hematological parameter could vary greatly in this group [29].

Hb H-CS disease is associated with increased amounts of Hb Bart's (γ₄) and Hb H inclusion bodies (β₄) [30]. Moreover, precipitation of unpaired β-globin chain was found in the erythropoietic cells from the bone marrow of Hb H patients [31]. A few studies reported that accumulation of the β-globin chain and α^{CS}-globin chain was mostly observed at the cytoskeleton part of the ghost red cell membrane [32, 33]. However, the more complex procedures of extracting the membrane associated proteins and peptides may undermine the purpose for high-throughput screening. Proteolytic peptides from the excess β-globin were identified even in peripheral blood, indicating the excess β-globin is also degraded by the endogenous proteases.

The proteolytic peptides detected in all the samples containing the α^{CS} allele originate from the elongated portion of the α-globin chain. There seems to be no cleavage in the normal sequence (α-globin 1-141). The shortened α^{CS} chains were detected by Weatherall and Clegg using starch-gel electrophoresis three decades ago [34]. Beside the full length α^{CS}, shortened α^{CS} chains were detected as CS₂ (1-169) and CS₃ (1-154). In the experiments described herein, the C-terminal tri-peptide cleavage resulted in CS₂ was not detected. The small peptide α^{CS170-172} could be lost during the Ziptip desalting procedure. The larger proteolytic peptide α^{CS155-172} could be further degraded from its N-terminal to yield smaller peptides detected in this report.

Amino acids in human proteins can be covalently modified by enzyme-catalyzed in vivo or chemical induced in vitro reactions. Modifications to arginine such as mono- and di-methylation might be related to pathophysiology of the thalassemic patient. In addition, arginine methylation is a common post-translational modification since protein arginine methyltransferase (PRMT) is a major component in eukaryotic cells. The role of arginine methylation has been implicated in RNA processing, transcriptional regulation, signal transduction, and DNA repair. However, cellular functions regulated by this modification in normal and diseased cells still remain to be clarified [35, 36].

### Example 2

The above LC/MS/MS experiments were repeated using a different LC system and a different database search engine, and the identified proteolytic disease fragments are listed in Tables 3 and 4. LC/MS/MS was used to generate these tables as this technique may provide a more comprehensive list of proteolytic peptides compared to MALDITOF. Sample preparation procedures for LC/MS remained unchanged as described in Example 1. LC/MS/MS procedures that differ from those described in Example 1 are described as follows.

A nano LC system (Dionex Ultimate 3000 RSLC, Sunnyvale, Calif., USA) was used for LC/MS. Purified peptide samples were injected onto a reverse phase column using the LC autosampler. The injection volume was 1-10 μl. A nano column (Dionex Acclaim PepMap RSLC, 75 μm ID, 15 cm, C18 2μm) was used for LC separation. A 30-min linear gradient (3-40% B) with mobile phases A (100% water, 0.1% formic acid) and B (100% acetonitrile, 0.1% formic acid) at a flow rate of 300 nl/min was used to elute peptides. The MS and MS/MS spectra were acquired with an LTQ-Orbitrap (Thermo, Bremen, Germany). The results were searched with Swiss-Prot protein database with added Hb CS and Hb E sequences using the Sequest search engine in the Trans Proteomic Pipeline or the ProLuCID search engine in the IP2 Integrated Proteomics Pipeline. Key search parameters were: enzyme specificity=none, MS accuracy=30 ppm, MS/MS accuracy=0.5 Da: Post-search filtering was done with peptide probability >0.95.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. As used in this specification and the appended claims, all ranges or lists as given are intended to convey any intermediate value or range or any sublist contained therein. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

[1] Anderson, N. L., Anderson, N. G., The human plasma proteome: history, character, and diagnostic prospects. Mol Cell Proteomics 2002, 1, 845-867.
[2] Liotta, L. A., Petricoin, E. F., Serum peptidome for cancer detection: spinning biologic trash into diagnostic gold. J Clin Invest 2006, 116, 26-30.
[3] Villanueva, J., Shaffer, D. R., Philip, J., Chaparro, C. A., et al., Differential exoprotease activities confer tumor-specific serum peptidome patterns. J Clin Invest 2006, 116, 271-284.
[4] Diamandis, E. P., Analysis of serum proteomic patterns for early cancer diagnosis: drawing attention to potential problems. J Natl Cancer Inst 2004, 96, 353-356.
[5] Paulovich, A. G., Whiteaker, J. R., Hoofnagle, A. N., Wang, P., The interface between biomarker discovery and clinical validation: The tar pit of the protein biomarker pipeline. Proteomics Clin. Appl. 2008, 2.
[6] Weatherall, D. J., Clegg, J. B., The Thalassaemia Syndrome, Blackwell Science Ltd., London 2001.
[7] Sanchaisuriya, K., Fucharoen, G., Fucharoen, S., Hb Pakse [(alpha2) codon 142 (TAA→TAT or Term→Tyr)] in Thai patients with EAbart's disease and Hb H Disease. Hemoglobin 2002, 26, 227-235.
[8] Pootrakul, P., Winichagoon, P., Fucharoen, S., Pravatmuang, P., et al., Homozygous haemoglobin Constant Spring: a need for revision of concept. Hum Genet 1981, 59, 250-255.
[9] Viprakasit, V., Tanphaichitr, V. S., Pung-Amritt, P., Petrarat, S., et al., Clinical phenotypes and molecular characterization of Hb H-Pakse disease. Haematologica 2002, 87, 117-125.
[10] Fucharoen, S., Winichagoon, P., Wisedpanichkij, R., Sae-Ngow, B., et al., Prenatal and postnatal diagnoses of thalassemias and hemoglobinopathies by HPLC. Clin Chem 1998, 44, 740-748.
[11] Singsanan, S., Fucharoen, G., Savongsy, O., Sanchaisuriya, K., Fucharoen, S., Molecular characterization and origins of Hb Constant Spring and Hb Pakse in Southeast Asian populations. Annals of hematology 2007, 86, 665-669.
[12] Brennan, S. O., Fifty-eight years of hemoglobin analysis. Clin Chem 2008, 54, 8-10.
[13] Wild, B. J., Green, B. N., Cooper, E. K., Lalloz, M. R., et al., Rapid identification of hemoglobin variants by electrospray ionization mass spectrometry. Blood Cells Mol Dis 2001, 27, 691-704.
[14] Rai, D. K., Griffiths, W. J., Landin, B., Wild, B. J., et al., Accurate mass measurement by electrospray ionization quadrupole mass spectrometry: detection of variants differing by <6 Da from normal in human hemoglobin heterozygotes. Anal Chem 2003, 75, 1978-1982.
[15] Rai, D. K., Green, B. N., Landin, B., Alvelius, G., Griffiths, W. J., Accurate mass measurement and tandem mass spectrometry of intact globin chains identify the low proportion variant hemoglobin Lepore-Boston-Washington from the blood of a heterozygote. J Mass Spectrom 2004, 39, 289-294.
[16] Daniel, Y. A., Turner, C., Haynes, R. M., Hunt, B. J., Dalton, R. N., Rapid and specific detection of clinically significant haemoglobinopathies using electrospray mass spectrometry-mass spectrometry. Br J Haematol 2005, 130, 635-643.
[17] Kleinert, P., Schmid, M., Zurbriggen, K., Speer, O., et al., Mass spectrometry: a tool for enhanced detection of hemoglobin variants. Clin Chem 2008, 54, 69-76.
[18] Veenstra, T. D., Global and targeted quantitative proteomics for biomarker discovery. J Chromatogr B Analyt Technol Biomed Life Sci 2007, 847, 3-11.
[19] Hortin, G. L., A new era in protein quantification in clinical laboratories: application of liquid chromatography-tandem mass spectrometry. Clin Chem 2007, 53, 543-544.
[20] Petricoin, E. F., Belluco, C., Araujo, R. P., Liotta, L. A., The blood peptidome: a higher dimension of information content for cancer biomarker discovery. Nat Rev Cancer 2006, 6, 961-967.
[21] Daniel, Y. A., Turner, C., Haynes, R. M., Hunt, B. J., Dalton, R. N., Quantification of hemoglobin A2 by tandem mass spectrometry. Clin Chem 2007, 53, 1448-1454.
[22] Pongsamart, S., Pootrakul, S., Wasi, P., Na-Nakorn, S., Hemoglobin Constant Spring: hemoglobin synthesis in heterozygous and homozygous states. Biochem Biophys Res Commun 1975, 64, 681-686.
[23] Derry, S., Wood, W. G., Pippard, M., Clegg, J. B., et al., Hematologic and biosynthetic studies in homozygous hemoglobin Constant Spring. J Clin Invest 1984, 73, 1673-1682.
[24] Williamson, D., The unstable haemoglobins. Blood reviews 1993, 7, 146-163.
[25] She, Y. M., Krokhin, O., Spicer, V., Loboda, A., et al., Formation of (bn-1+H2O) ions by collisional activation of MALDI-formed peptide [M+H]+ ions in a QqTOF mass spectrometer. J Am Soc Mass Spectrom 2007, 18, 1024-1037.
[26] Charoenkwan, P., Sirichotiyakul, S., Chanprapaph, P., Tongprasert, F., et al., Anemia and hydrops in a fetus with homozygous hemoglobin constant spring. J Pediatr Hematol Oncol 2006, 28, 827-830.
[27] Clarke, G. M., Higgins, T. N., Laboratory investigation of hemoglobinopathies and thalassemias: review and update. Clinical chemisby 2000, 46, 1284-1290.
[28] Chui, D. H., Alpha-thalassemia: Hb H disease and Hb Barts hydrops fetalis. Annals of the New York Academy of Sciences 2005, 1054, 25-32.
[29] Sanchaisuriya, K., Fucharoen, G., Sae-ung, N., Jetsrisuparb, A., Fucharoen, S., Molecular and hematologic features of hemoglobin E heterozygotes with different forms of alpha-thalassemia in Thailand. Annals of hematology 2003, 82, 612-616.
[30] Chui, D. H., Fucharoen, S., Chan, V., Hemoglobin H disease: not necessarily a benign disorder. Blood 2003, 101, 791-800.
[31] Wickramasinghe, S. N., Lee, M. J., Evidence that the ubiquitin proteolytic pathway is involved in the degradation of precipitated globin chains in thalassaemia. British journal of haematology 1998, 101, 245-250.
[32] Schrier, S. L., Bunyaratvej, A., Khuhapinant, A., Fucharoen, S., et al., The unusual pathobiology of hemoglobin constant spring red blood cells. Blood 1997, 89, 1762-1769.
[33] Advani, R., Sorenson, S., Shinar, E., Lande, W., et al., Characterization and comparison of the red blood cell membrane damage in severe human alpha- and beta-thalassemia. Blood 1992, 79, 1058-1063.
[34] Weatherall, D. J., Clegg, J. B., The alpha-chain-termination mutants and their relation to the alpha-thalassaemias. Philos Trans R Soc Lond B Biol Sci 1975, 271, 411-455.
[35] Pahlich, S., Zakaryan, R. P., Gehring, H., Protein arginine methylation: Cellular functions and methods of analysis. Biochimica et biophysica acta 2006, 1764, 1890-1903.
[36] Bedford, M. T., Richard, S., Arginine methylation an emerging regulator of protein function. Molecular cell 2005, 18, 263-272.
[37] Vichinsky, E., Hemoglobin E syndromes. Hematology 2007, 79-83.
[38] Yuan, J., Bunyaratvei, A., Fucharoen, S., Fung, C., Shinar, E., Schrier, S. L., The instability of the membrane skeleton in thalassemic red blood cells. Blood 1995, 86, 3945-3950.

1. A method of detecting a biomarker for a hemoglobinopathy or thalassemia genetic disease in an individual, the method comprising:
identifying the presence of a proteolytic peptide in a polypeptide fraction, the polypeptide fraction obtained from a cellular extract of cells of the individual, which cells are affected by the disease, the proteolytic peptide containing a mutation associated with the genetic disease or directly related to the disease condition, the proteolytic peptide consisting of a sequence set forth in any one of SEQ ID NOs. 6-201.

2. The method of claim 1, wherein the mutation comprises an alteration in post-translational modification as compared to a non-disease proteolytic peptide.

3. The method of claim 2, wherein the post-translational modification comprises dimethylation of R166.

4. The method of claim 1, further comprising separating the polypeptide fraction from cellular debris in the cellular extract prior to the identifying.

5. The method of claim 1, further comprising lysing cells from the individual that are affected by the disease to obtain the cellular extract.

6. The method of claim 1, wherein the identifying comprises mass spectrometry.

7. The method of claim 6, wherein the mass spectrometry comprises MALDI-TOF, LC/MS/MS, high resolution LC/MS, MRM or SRM techniques.

8. The method of claim 1, wherein the cells affected by the disease are red blood cells.

9. The method of claim 1, further comprising lysing cells from the individual that are affected by the disease to obtain the cellular extract and separating the polypeptide fraction from cellular debris in the cellular extract prior to the identifying, wherein the mutation comprises an alteration in post-translational modification that is dimethylation of R166 and the identifying comprises mass spectrometry.
